(19) United States
(12) Patent Application Publication
(10) Pub. No.: US 2003/0150027 A1
(43) Pub. Date: Aug. 7, 2003

(54) Transgenic plants with increased seed yield, biomass and harvest index

(76) Inventor: Michael Giroux, Bozeman, MT (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/116,868

(22) Filed: Apr. 5, 2002

Related U.S. Application Data
(63) Continuation of application No. 09/516,250, filed on Mar. 1, 2000, now abandoned.

(51) Int. Cl. A01H 5/00; C12N 15/82

(52) U.S. Cl. 800/287; 800/320.1; 800/320.2; 800/320.3

(57) ABSTRACT

This invention provides methods for producing plants with increased seed and biomass production. More specifically, this invention provides methods for producing plants which have increased yields for a number of plant traits, including seed number, seed weight, the number of seed heads, flag leaf weight and total plant weight. This invention also provides methods for improving the Harvest Index of a plant.

### CROSS-REFERENCE TO RELATED APPLICATION

This application is a continuation of U.S. application Ser. No. 09/516,250, filed Mar. 1, 2000.

### FIELD OF THE INVENTION

This invention relates to improving plant production, both plant seed production and plant biomass production. More specifically, this invention relates to transgenic plants which have increased seed production and increased biomass production when compared to non-transgenic plants of the same genetic background. Even more specifically, this invention relates to plants which are transgenic for Sh2−Rev6-HS and to methods for producing such plants.

### BACKGROUND OF THE INVENTION

All publications and patent applications herein are incorporated by reference to the same extent as if each individual publication or patent application was specifically and individually indicated to be incorporated by reference.

ADP glucose pyrophosphorylase (AGP) is one of the primary enzymes involved in the biosynthesis of starch and glycogen in organisms such as plants, algae, fungi and bacteria, particularly plants. AGP catalyzes the following reaction:

α-glucose-1-P+ATPADP-glucose+PP₁.

ADP-glucose, the product of the above reaction, is the major donor of glucose in the biosynthesis of starch in plants and in the biosynthesis of glycogen by bacteria.

AGP is widely distributed throughout the plant kingdom. It is present in monocots such as wheat, rice, barley, and maize, as well as dicots such as spinach, potato, and pea. It is also found in some starch producing bacteria, such as E. coli. Plant AGP exists as a tetramer (210 to 240 kDa) composed of two small sub-units (50 to 55 kDa) and two large sub-units (51 to 60 kDa) in contrast to bacterial AGP which appears to consist of four units of equal size. AGP has also been shown to be produced in cyanobacteria and in algae, where its tetrameric structure is similar to that in plants, i.e. two large and two small sub-units, rather than the homotetrameric structure found in ordinary bacteria.

Because of the commercial importance of starch, primarily as a foodstuff but also as an important industrial chemical, much work has been done to isolate and characterize the nucleic acid encoding AGP. Plant AGP consists of two different protein subunits. In maize endosperm, AGP is encoded by the Shrunken-2 (Sh2) and Brittle-2 (Bt2) genes (Bhave et al., 1990 and Bae et al., 1990). Sh2 encodes the large subunit having a predicted molecular weight of 57,179 Da, while Bt2 encodes the small subunit having a molecular weight of 52,224 Da. The isolation of nucleic acids encoding AGPs from various other plants has also been reported: the small subunit cDNA (Anderson et al., 1989) and the genomic DNA (Anderson et al., 1991) from rice; the small and large subunit cDNAs from spinach leaf (Morell et al., 1988); and the small and large subunit cDNAs from potato tuber (Muller-Rober et al., 1990; and Nakata et al., 1991).

Moreover, work has been done to alter AGP expression in plants in order to regulate starch synthesis. EP 455,316 provides a plasmid that comprises a DNA encoding AGP placed in an inverted orientation, which results in the transcription of the anti-sense mRNA in a host plant. The patent shows that transgenic potatoes comprising the plasmid has reduced AGP activity and reduced starch concentration compared to non-transformed plants. U.S. Pat. No. 5,773,693 discloses a method of increasing sucrose content of pea plant by suppressing or reducing the expression of either or both subunits of AGP. The method comprises transforming a pea plant with a plasmid comprising nucleic acid encoding the Sh2 subunit or the Bt2 or both subunits in antisense orientation to the promoter and the terminator.

In contrast, U.S. Pat. No. 5,977,437 teaches a method of increasing the rate and/or yield of starch production in a plant comprising introducing into a plant, a nucleic acid encoding barley endosperm AGP operably linked to a plastid transit peptide. EP 634,491 discloses a method of decreasing oil content in seed by increasing the amount of starch comprising transforming a plant cell with a nucleic acid comprising a promoter, and a DNA encoding a fusion protein comprising an amino terminal plastid transit peptide, an AGP enzyme, and a 3′ non-translated transcription termination sequence, obtaining transformed plant cells, and regenerating transformed plants from the transformed plant cells. Finally, U.S. Pat. No. 5,792,290 discloses the nucleic acid encoding wheat AGP and teaches inserting extra copies of the AGP gene into a plant genome by transformation to enhance starch production and inserting the complement of the mRNA encoding the endogenous AGP to reduce starch production.

The maize endosperm is the site of most starch deposition during kernel development. Sh2 and Bt2 maize endosperm mutants have greatly reduced starch levels corresponding to deficient levels of AGP activity. Mutations of either gene have been shown to reduce AGP activity by about 95% (Tsai et al., 1966; Dickinson et al., 1969). Lack of AGP and a decrease in starch levels compared to that of the wild-type endosperm result in shrunken, brittle, and/or collapsed kernels at seed maturity. Furthermore, it has been observed that enzymatic activities increase with the dosage of functional wild-type Sh2 and Bt2 alleles, whereas mutant enzymes have altered kinetic properties.

AGP is the rate limiting step in starch biosynthesis in plants. Stark et al. placed a mutant form of E. coli AGP in potato tuber and obtained a 35% increase in starch content (Stark et al., 1992). AGP is an allosteric enzyme, i.e. its activity is regulated through the binding of an effector to an allosteric site. In plants, the positive effector of AGP is 3-phosphoglycerate (3-PGA), and the negative effector is phosphate (Dickinson et al., 1969). Inhibition of AGP by phosphate is likely the largest limitation on starch biosynthesis in plants (Giroux et al., 1996).

Giroux et al. (1996; U.S. Pat. Nos. 5,872,216 and 5,589,618, each of which is hereby incorporated by reference in their entireties) used in vivo, site-specific mutagenesis to create short insertion mutations in a region of the gene known to be involved in the allosteric regulation of AGP. Single mutations of the Sh2 gene containing an insertion of an additional tyrosine or serine residue reduced total AGP activity and the amount of SH2 protein. A specific revertant containing an additional tyrosine residue and an additional serine residues increased seed weight 11-18%. This later revertant were named “Sh2−m1Rev6” (this gene is designated as “Sh2−Rev6” herein). Giroux et al. (1996) also found that the increase in seed weight of the Sh2−m1Rev6 was not solely attributable to an increase in starch content, although there was an increase in the absolute starch content in the variant expressing Sh2-m1Rev6. Giroux et al. (1996) suggested that the enhanced starch synthesis caused by Rev6 creates a stronger sink within the seed leading to increased synthesis of other seed components.

Modulation of the sink strength of a plant is one of the methods to increase harvest yield. The leaves and other green tissue active in photosynthesis are commonly referred to as the “source,” and those parts in which storage occurs are referred to as the “sink”. In cereals such as maize, rice, and wheat, the primary sink is the endosperm, and individual seed weight is the primary determinant of the yield of corn (Duvick et al., 1992). As evidenced by Giroux et al. (1996), rendering the maize endosperm AGP insensitive to phosphate inhibition, increases individual seed weight without dramatically affecting starch content (U.S. Pat. Nos. 5,650,557 and 5,872,216).

Over the years, the desire for high biological yield has aroused an interest in manipulating plant structure in order to obtain plants where the economically useful part forms are as large a proportion of the plant as is consistent with acceptable plant vigor and health. Attempts to increase yield by altering the relative contribution of the different components of grain or kernel yield, such as ears or heads per plant, grains per head or kernels per ear, grain size or kernel size, etc., have proven unsuccessful because increases in one component tend to be accompanied by reductions in another (Wilson, D. (1981) Plant Breeding II. K. Frey Edited, Iowa, Iowa State University Press, page 255). However, yield increases due to an increase in the proportion of grain relative to vegetative parts have been common in the cereal crops (Wilson, D. (1981) Plant Breeding II. K. Frey Edited, Iowa, Iowa State University Press, page 255).

Langer and Hill (Langer, R. H. M. and Hill, G. D. (1991) Agricultural Plants. Second Edition. Cambridge, Cambridge University Press, page 341) state that higher yields can be achieved by improving the Harvest Index (HI), since HI links biological yield (Y_{biol}) and economic yield (Y_{econ}) in the following manner:

Y_{biol×HI=Y}_{econ}.

It is pointed out that treatments affecting HI will also affect Y_{biol} but not necessarily to the same extent or in the same direction. For instance, in cereals it is possible to increase biological yields by applying nitrogen at high population densities in the presence of adequate water. The expected result is heavy vegetative growth, but reduced light transmission into the canopy, poor grain set and development will lead to a low Harvest Index. In contrast, short-strawed cereals are characterized by greater Harvest Index. Short, erect cultivars of rice yielding 4-5 t ha⁻¹ have been shown to have a Harvest Index of about 0.53 to 0.56, compared with 0.39 to 0.42 for tall, leafy cultivars with a grain yield of about 2.4 t ha⁻¹ (Langer, R. H. M. and Hill, G. D. (1991) Agricultural Plants. Second Edition, Cambridge, Cambridge University Press, page 341). Likewise, in wheat, the dwarf and semidwarf cultivars emanating from the Mexican plant breeding program have a higher Harvest Index. However, short plants may also produce little grain. Thus, it is necessary to assess both the biological yield and Harvest Index in plant breeding programs.

The present invention provides a method of increasing the seed production and the biomass production of plants. More specifically, this invention provides transgenic plants which have increased total seed number, increased individual seed weight, increased total seed weight per plant, as well as increased above-ground plant biomass and an improved Harvest Index when compared to non-transgenic plants of the same genetic background. The production of plants with increases in all of these parameters as the result of a transgene is quite unexpected in view of normal source/sink relationships in plants.

### BRIEF SUMMARY OF THE INVENTION

This invention provides methods of producing plants which have improved plant production, both plant seed production and plant biomass production. This invention also provides the plants produced by the disclosed methods, wherein the plants are monocotyledonous plants and dicotyledonous plants.

More specifically, this invention provides methods for increasing the number of seeds produced by plants, increasing the biomass produced by plants, or increasing the Harvest Index of plants by introducing into such plants a nucleic acid operably linked to a promoter, wherein the nucleic acid is that of SH2−REV6-HS (SEQ ID NO: 3), a nucleic acid which hybridizes with SH2−REV6-HS under high stringency conditions and encodes a polypeptide that retains biological activity of the protein SH2−REV6-HS (SEQ ID NO: 4), or a fragment of SH2−REV6-HS encoding a peptide that retains biological activity of SH2−REV6-HS. The methods further include growing the plants produced by such methods. The present invention also includes the plants produced by such methods.

The methods of the present invention are applicable to monocotyledonous plants, such as rice or wheat, and dicotyledonous plants, such as peas.

This invention also provides methods of increasing the flag leaf weight of monocotyledonous plants by introducing into such plants a nucleic acid operably linked to a promoter, wherein the nucleic acid is that of SH2−REV6-HS (SEQ ID NO: 3), a nucleic acid which hybridizes with SH2−REV6-HS under high stringency conditions and encodes a polypeptide that retains biological activity of the protein SH2−REV6-HS (SEQ ID NO: 4), or a fragment of SH2−REV6-HS encoding a peptide that retains biological activity of SH2-REV6-HS. The methods further include growing the plants produced by such methods. The present invention also includes the plants produced by such methods. The invention also provides methods of increasing the number of seed heads produced by monocotyledonous plants by introducing into such plants a nucleic acid operably linked to a promoter, wherein the nucleic acid is that of SH2−REV6-HS (SEQ ID NO: 3), a nucleic acid which hybridizes with SH2−REV6-HS under high stringency conditions and encodes a polypeptide that retains biological activity of the protein SH2−REV6-HS (SEQ ID NO: 4), or a fragment of SH2−REV6-HS encoding a peptide that retains biological activity of SH2-REV6-HS. The methods further include growing the plants produced by such methods. The present invention also includes the plants produced by such methods.

The invention also provides methods of increasing two or more traits of dicotyledonous plants by introducing into such plants a nucleic acid operably linked to a promoter, wherein the nucleic acid is that of SH2−REV6-HS (SEQ ID NO: 3), a nucleic acid which hybridizes with SH2−REV6-HS under high stringency conditions and encodes a polypeptide that retains biological activity of the protein SH2−REV6-HS (SEQ ID NO: 4), or a fragment of SH2−REV6-HS encoding a peptide that retains biological activity of SH2-REV6-HS. The methods further include growing the plants produced by such methods. The present invention also includes the plants produced by such methods.

The invention further provides methods of increasing the yield of two or more traits of monocotyledonous plants by introducing into such plants a nucleic acid operably linked to a promoter, wherein the nucleic acid is that of SH2−REV6-HS (SEQ ID NO: 3), a nucleic acid which hybridizes with SH2−REV6-HS under high stringency conditions and encodes a polypeptide that retains biological activity of the protein SH2−REV6-HS (SEQ ID NO: 4), or a fragment of SH2−REV6-HS encoding a peptide that retains biological activity of SH2-REV6-HS. The methods further include growing the plants produced by such methods. The present invention also includes the plants produced by such methods.

The present invention further includes crossing the plants obtained by the above-methods to one or more other plants and harvesting and growing the seed which is produced as a result of making the crosses.

The present invention further includes harvesting seed which produced by selling the plants obtained by the above methods and growing the harvested seed.

The present invention provides plants which include a nucleic acid encoding the amino acid sequence of SH2−REV6-HS (SEQ ID NO: 4).

### BRIEF DESCRIPTION OF THE SEQUENCES

SEQ ID NO: 1 is a nucleotide sequence of Sh2−Rev6 that can be used according to the subject invention.

SEQ ID NO: 2 is the amino acid sequence encoded by the nucleotide sequence of SEQ ID NO. 1 that can be used according to the subject invention.

SEQ ID NO: 3 is a nucleotide sequence of Sh2−Rev6-HS that can be used according to the subject invention.

SEQ ID NO: 4 is the amino acid sequence encoded by the nucleotide sequence of SEQ ID NO. 3 that can be used according to the subject invention.

SEQ ID NO: 5 is a PCR primer that can be used according to the subject invention.

SEQ ID NO: 6 is a PCR primer that can be used according to the subject invention.

### DETAILED DESCRIPTION OF THE INVENTION

I. Definitions

As used herein, the term “AGP” means ADP glucose pyrophosphorylase.

As used herein, the term “allele” means any of several alternative forms of a gene.

As used herein, the term “biological activity” means any functional activity of the SH2−REV6-HS polypeptide. The functional activity of the SH2−REV6-HS polypeptide includes but are not limited increasing total seed number, increasing individual seed weight, increasing total seed weight per plant, increasing above-ground plant biomass, and increasing Harvest Index.

As used herein, the term “Bt2” means the Brittle-2 gene encoding the small subunit of AGP. As used herein, the term “bt2” means a mutant form of the Bt2 gene, which renders the kernels of corn brittle in texture upon drying.

As used herein, the term “cereal” means, depending on the context, either: 1) a grass plant, such as corn, or 2) the grain of a grass plant.

As used herein, the term “crop plant” means any plant grown for any commercial purpose, including, but not limited to the following purposes: seed production, grain production, hay production, ornamental use, fruit production, berry production, vegetable production, oil production, protein production, forage production, silage, animal grazing, golf courses, lawns, flower production, landscaping, erosion control, green manure, improving soil tilth/health, producing pharmaceutical products/drugs, producing food additives, smoking products, pulp production and wood production. Particular crop plants of interest to the present invention include, but are not limited to, wheat, rice, maize, barley, rye, sugar beets, potatoes, sweet potatoes, soybeans, cotton, tomatoes, canola and tobacco.

As used herein, the term “cross pollination” or “cross-breeding” means the pollen of one flower on one plant is applied (artificially or naturally) to the ovule (stigma) of a flower on another plant.

As used herein, the term “cultivar” means a variety, strain or race of plant which has been produced by horticultural or agronomic techniques and is not normally found in wild populations.

As used herein, the terms “Dicotyledoneae,” “dicotyledonous,” “dicotyledon” or “dicot” are synonymous and mean any of various flowering plants having two embryonic seed leaves or cotyledons that usually appear at germination. Examples include, but are not limited to, tobacco, soybeans, potato, sweet potato, radish, cabbage, rape and apple trees.

As used herein, the term “flag leaf” refers to the uppermost leaf on a fruiting (fertile) culm; the leaf immediately below the inflorescence or seed head.

As used herein, the term “genotype” means the genetic makeup of an individual cell, cell culture, plant, or group of plants.

As used herein, the term “grain” means, depending on its context, either: 1) the cereal grasses considered as a group, or 2) the fruit of one or more of the cereal grasses.

As used herein, the terms “grass” or “grasses” mean a plant belonging to the family Poaceae.

As used herein, the term “Harvest Index” is the proportion of total plant mass harvested. It is the ratio of weight of grain/(weight of grain plus weight of plant). This is identical to HI as discussed elsewhere herein (see, also, Langer and Hill, 1991), wherein HI links biological yield and economic yield, and HI is the ratio of economic yield/biological yield. The economic yield (Y_{econ}) is the weight of grain, while the biological yield (Y_{biol}) is the weight of grain plus weight of plant. The weight of grain is synonymous with the total seed weight.

As used herein, the term “heterozygote” means a diploid or polyploid individual cell or plant having different alleles (forms of a given gene) at least at one locus.

As used herein, the term “heterozygous” means the presence of different alleles (forms of a given gene) at a particular gene locus.

As used herein, the term “homozygote” means an individual cell or plant having the same alleles at one or more loci.

As used herein, the term “homozygous” means the presence of identical alleles at one or more loci in homologous chromosomal segments.

As used herein, the term “hybrid” means any individual plant resulting from a cross between parents that differ in one or more genes.

As used herein, the term “inbred” or “inbred line” means a relatively true-breeding strain.

As used herein, a nucleic acid molecule is said to be “isolated” when the nucleic acid molecule is substantially separated from contaminant nucleic acid encoding other polypeptides from the source of nucleic acid.

As used herein, the term “line,” when directed to a type of plant, means self- or cross-fertilizing plants and single-line facultative apomicts, having largely the same genetic background, that are similar in essential and distinctive characteristics.

As used herein, the term “locus” (plural: “loci”) means any site that has been defined genetically. A locus may be a gene, or part of a gene, or a DNA sequence that has some regulatory role, and may be occupied by different sequences.

As used herein, the term “mass selection” means a form of selection in which individual plants are selected and the next generation propagated from the aggregate of their seeds.

As used herein, the terms “Monocotyledoneae,” “monocotyledonous,” “monocotyledon” or “monocot” are synonymous and mean any of various flowering plants having a single cotyledon in the seed. Examples of monocots include, but are not limited to, rice, wheat, barley, maize and lilies.

As used herein, the term “Northern Blot” refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook et al., Molecular Cloning: A Laboratory Manual, 2nd edition, Cold Spring Harbor Laboratory Press, 1985).

As used herein, the term “open pollination” means a plant population that is freely exposed to some gene flow, as opposed to a closed one in which there is an effective barrier to gene flow.

As used herein, the terms “open-pollinated population” or “open-pollinated variety” mean plants normally capable of at least some cross-fertilization, selected to a standard, that may show variation but that also have one or more genotypic or phenotypic characteristics by which the population or the variety can be differentiated from others. A hybrid which has no barriers to cross-pollination is an open-pollinated population or an open-pollinated variety.

As used herein, the term “ovule” means the female gametophyte, whereas the term “pollen” means the male gametophyte.

As used herein, the term “phenotype” means the observable characters of an individual cell, cell culture, plant, or group of plants which results from the interaction between that individual's genetic makeup (i.e., genotype) and the environment.

As used herein, the term “progeny” means the descendants of a particular plant (self-cross) or pair of plants (crossed or backcrossed). The descendants can be of the F₁, the F₂, or any subsequent generation. Typically, the parents are the pollen donor and the ovule donor which are crossed to make the progeny plant of this invention. Parents also refer to F₁ parents of a hybrid plants of this invention (the F₂ plants). Finally, parents refer to a recurrent parent which is backcrossed to hybrid plants of this invention to produce another hybrid plant of this invention.

As used herein, the term “Polymerase Chain Reaction” is synonymous with “PCR” and refers to techniques in which cycles of denaturation, annealing with primer, and extension with DNA polymerase, are used to amplify the number of copies of a target DNA sequence.

As used herein, the term “revertant” refers to a mutated Sh2 gene (i.e., mutated relative to the wild-type Sh2 gene) wherein the mutant results in a wild-type kernel phenotype (i.e., a plump seed, not a shrunken seed like the phenotype displayed by the mutant sh2sh2 genotype). A revertant genotype would have more AGP activity than a sh2sh2 genotype and may have either more or less AGP activity than a wild-type Sh2 genotype. Typically, the revertants have a wild-type seed phenotype with at least around 30% AGP activity compared to that of a normal (i.e., non-revertant), wild-type. In some instances, the term “revertant” may refer to the cell or plant which contains the mutated Sh2 gene.

As used herein, the term “rice” means any Oryza species, including, but not limited to, O. sativa, O. glaberrima, O. perennis, O. nivara, and O. breviligulata. Thus, as used herein, the term “rice” means any type of rice including, but is not limited to, any cultivated rice, any wild rice, any rice species, any intra- and inter-species rice crosses, all rice varieties, all rice genotypes and all rice cultivars.

As used herein, the term “self pollinated” or “self-pollination” means the pollen of one flower on one plant is applied (artificially or naturally) to the ovule (stigma) of the same or a different flower on the same plant.

As used herein, the term “Sh2” refers to the Shrunken-2 gene encoding the large subunit of AGP. Sometimes, the term may refer to the cell or plant which contains the Sh2 genotype.

As used herein the term “sh2” means a mutant form of the Sh2 gene, which renders the kernels of corn shrunken or collapsed upon drying. Sometimes, the term may refer to the cell or plant which contains the sh2 genotype.

As used herein, the term “Sh2hs” refers to mutants of the Shrunken-2 gene which encode heat-stable variants of maize endosperm AGP. Sometimes, the term may refer to the cell or plant which contains the Sh2hs genotype.

As used herein, the term “Sh2hs33” refers to a single point mutation in Sh2 which increases the stability of maize endosperm AGP through enhanced subunit interactions. The mutation is a change from His-to-Tyr at amino acid position 333 (Greene and Hannah, 1998). Sometimes, the term may refer to the cell or plant which contains the Sh2hs33 genotype.

As used herein, the term “Sh2−Rev6” is synonymous with “Sh2−ml-Rev6” and refers to a variants of the Shrunken-2 gene. The polypeptide product of the Sh2−Rev6 gene contains two additional amino acids, tyrosine and serine, inserted between amino acids 494 and 495 of the wild-type Sh2 polypeptide. Maize endosperm encoded by Sh2−Rev6 expresses an AGP that is insensitive to phosphate and results in an increased seed weight in maize (Giroux et al., 1996; U.S. Pat. Nos. 5,650,557, and 5,872,216). Sometimes, the term may refer to the cell or plant which contains the Sh2−Rev6 genotype.

As used herein, the term “Sh2−Rev6-HS” is synonymous with “Sh2−m1Rev6-HS” and refers to a heat stable variant of the Sh2−Rev6 gene, wherein His is replaced by Tyr at position 333. Sometimes, the term may refer to the cell or plant which contains the Sh2−Rev6-HS genotype.

As used herein, the term “Sh2hs33” refers to a specific heat stable genetic variant of Sh2. The variant contains a His to Tyr mutation at position 333 of the wild-type maize Sh2 gene (Greene and Hannah, 1998). The mutation renders the maize endosperm AGP activity heat-stable. Sometimes, the term may refer to the cell or plant which contains the Sh2hs33 genotype.

As used herein, the phrase “shrunken and brittle” describes the morphology of specific types of kernels of a corn. In a brittle and shrunken kernel, the endosperm is greatly collapsed. The endosperm before drying is like a fluid-filled sac that develops with little starch. On drying, the kernel shrinks and collapses into an angular structure with marked concavities and brittle texture (Coe et al., 1988).

As used herein, the term “synthetic” means a set of progenies derived by intercrossing a specific set of clones or seed-propagated lines. A synthetic may contain mixtures of seed resulting from cross-, self-, and sib-fertilization.

As used herein, the terms “T₁, T₂, T₃, . . . ” refer to the succeeding generations of cells or plants tracing back to a particular tissue culture-derived or transformed cell line designated as T₀, or the parental generation. As regards plants, the plants produced directly from the transformed cells are referred to as the T₀ generation. The seeds produced by selfing the T₀ generation plants are referred to as the T₁ seeds. When the T₁ seeds are germinated, the resulting plants are referred to as the T₁ generation or the T₁ progeny. Seeds produced by the T₁ generation are referred to as the T₂ seeds.

As used herein, in grasses, the term “tiller” means a lateral shoot arising at ground level. Each of the tillers that were counted in the present studies had a head on the stem of the shoot.

As used herein, the term “transformation” means the transfer of nucleic acid (i.e., a nucleotide polymer) into a cell. As used herein, the term “genetic transformation” means the transfer and incorporation of DNA, especially recombinant DNA, into a cell.

As used herein, the term “transgenic” means cells, cell cultures, plants, and progeny of plants which have received a foreign or modified nucleic acid sequence by one of the various methods of transformation, wherein the foreign or modified nucleic acid sequence is from the same or different species than the species of the plant receiving the foreign or modified nucleic acid sequence. The foreign or modified nucleic acid used to produce such transgenic cells, cell cultures, plants and progeny of such plants includes genes, gene fragments as well as nucleic acid sequences which code for a product which has at least one biological activity or function. As used herein, the terms “transgenic plant” and “transformed plant” are synonymous, as are the terms “transgenic line” and “transformed line”. As used herein, the phrases “corresponding non-transgenic plant” and “corresponding non-transgenic line” refer to the cells, cell cultures, plants and progeny of plants which did not receive the foreign or modified gene which the “transgenic” cells, cell cultures, plants and progeny of plants which did receive the foreign or modified gene.

As used herein, the term “variety” means a subdivision of a species, consisting of a group of individuals within the species which are distinct in form or function from other similar arrays of individuals.

As used herein, the term “wheat” means any Triticum species, including, but not limited to, T. aestivum, T. monococcum, T. tauschii and T. turgidum. Thus, as used herein, the term “wheat” means any type of wheat including, but is not limited to, any cultivated wheat, any wild wheat, any wheat species, any intra- and inter-species wheat crosses, all wheat varieties, all wheat genotypes and all wheat cultivars. Cultivated wheats include, but are not limited to, einkorn, durum and common wheats.

As used herein, the term “wild-type” refers to the naturally occurring allele of a particular gene. Sometimes the terms refers to the cell or plant containing the wild-type alleles of the particular gene.

II. Nucleic Acids Encoding Sh2−Rev6 and Sh2−Rev6-HS

Giroux et al. (1996) isolated and sequenced genomic DNA and cDNA encoding Sh2-Rev6. The nucleotide sequence of Sh2−Rev6 is provided in SEQ ID NO: 1 and the amino acid sequence of SH2−REV6 is provided in SEQ ID NO: 2 (see, also, U.S. Pat. No. 5,650,557 and U.S. Pat. No. 5,872,216). Corn seeds that contain at least one functional Sh2−Rev6 allele have been deposited with the American Type Culture Collection (ATCC), 12301 Parklawn Drive, Rockville, Md., 20852 USA, on May 16, 1999 and assigned accession number ATCC 97624 (see, column 5 of U.S. Pat. Nos. 5,650,557 and 5,872,216).

Sh2−Rev6 was further modified by altering His to Tyr at amino acid position 333 to produce the variant Sh2−Rev6-HS (Giroux et al. (1998)). The nucleotide sequence of Sh2-Rev6-HS is provided in SEQ ID NO: 3 and the amino acid sequence of SH2−REV6-HS is provided in SEQ ID NO: 4.

As used herein, Sh2−Rev6 and Sh2−Rev6-HS include the specifically identified and characterized variants herein described as well as allelic variants, conservative substitution variants and homologues that can be isolated/generated and characterized without undue experimentation following methods well known to one skilled in the art.

Homology or identity at the amino acid or nucleotide level is determined by BLAST (Basic Local Alignment Search Tool) analysis using the algorithm employed by the programs blastp, blastn, blastx, tblastn and tblastx (Karlin et al., 1990, Proc. Natl. Acad. Sci. U.S. Pat. No. 87,2264-2268 and Altschul, 1993, J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases (see Altschul et al., 1994, Nature Genetics 6, 119-129 which is fully incorporated by reference). The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., 1992, Proc. Natl. Acad. Sci. USA 89, 10915-10919, fully incorporated by reference). For blastn, the scoring matrix is set by the ratios of M (i.e., the reward score for a pair of matching residues) to N (i.e., the penalty score for mismatching residues), wherein the default values for M and N are 5 and -4, respectively.

The terms “Sh2−Rev6 genes” and “Sh2−Rev6-HS genes” include all allelic variants of the Sh2−Rev6 genes and Sh2−Rev6-HS genes exemplified herein, wherein such allelic variants code for proteins which result in one or more of the same physiological characteristics as those of the proteins produced by the Sh2−Rev6 and Sh2−Rev6-HS genes disclosed herein.

The Sh2−Rev6 and Sh2−Rev6-HS nucleic acid molecules or fragments thereof utilized in the present invention may also be synthesized using methods known in the art. It is also possible to produce the molecule by genetic engineering techniques, by constructing DNA using any accepted technique, cloning the DNA in an expression vehicle and transfecting the vehicle into a cell which will express the SH2−REV6 and SH2−REV6-HS proteins. See, for example, the methods set forth in Sambrook et al., Molecular Cloning: A Laboratory Manual, 2nd edition, Cold Spring Harbor Laboratory Press, 1985.

It is understood that all polynucleotides encoding all or a portion of the SH2−REV6 and SH2−REV6-HS proteins used in the present invention are also included herein, as long as they encode a polypeptide with one or more of the functional activities of the SH2−REV6 and SH2−REV6-HS proteins as set forth herein. Thus, any polynucleotide fragment having the activities of the SH2−REV6 and SH2−REV6-HS proteins discussed herein are encompassed by the present invention.

Polynucleotide sequences of the invention include DNA, cDNA, synthetic DNA and RNA sequences which encode SH2−REV6 and SH2−REV6-HS proteins. Such polynucleotides also include naturally occurring, synthetic and intentionally manipulated polynucleotides. For example, such polynucleotide sequences may include genomic DNA which may or may not include naturally occurring introns. Moreover, such genomic DNA may be obtained in association with promoter regions or poly A sequences. As another example, portions of the mRNA sequence may be altered due to alternate RNA splicing patterns or the use of alternate promoters for RNA transcription. As yet another example, Sh2−Rev6 and Sh2−Rev6-HS polynucleotides may be subjected to additional site-directed mutagenesis.

The polynucleotides of the invention further include sequences that are degenerate as a result of the genetic code. The genetic code is said to be degenerate because more than one nucleotide triplet codes for the same amino acid. There are 20 natural amino acids, most of which are specified by more than one codon. It will be appreciated by those skilled in the art that as a result of the degeneracy of the genetic code, a multitude of nucleotide sequences, some bearing minimal nucleotide sequence homology to the nucleotide sequence of Sh2−Rev6 and Sh2−Rev6-HS may be utilized in the present invention. Therefore, all degenerate nucleotide sequences are included in the invention as long as the amino acid sequence of the SH2−REV6 and SH2−REV6-HS polypeptides encoded by the nucleotide sequence are functionally unchanged or substantially similar in function. The invention specifically contemplated each and every possible variation of peptide or nucleotide sequence that could be made by selecting combinations based on the possible amino acid and codon choices made in accordance with the standard triplet genetic code as applied to the Sh2−Rev6 and Sh2-Rev6-HS sequences of the invention, as exemplified by Sh2−Rev6 and Sh2−Rev6-HS, and all such variations are to be considered specifically disclosed herein.

Also included in the invention are fragments (portions, segments) of the sequences disclosed herein which selectively hybridize to Sh2−Rev6 and Sh2−Rev6-HS. Selective hybridization as used herein refers to hybridization under stringent conditions (See, for example, the techniques in Maniatis et al. (1989) Molecular Cloning: A Laboratory Manual, Cold Spring Harbor Laboratory Press), which distinguishes related from unrelated nucleotide sequences. The active fragments of the invention, which are complementary to mRNA and the coding strand of DNA, are usually at least about 15 nucleotides, more usually at least 20 nucleotides, preferably 30 nucleotides and more preferably may be 50 nucleotides or more.

“Stringent conditions” are those that (1) employ low ionic strength and high temperature for washing, for example, 0.5 M sodium phosphate buffer pH 7.2, 1 mM EDTA pH 8.0 in 7% SDS at either 65° C. or 55° C., or (2) employ during hybridization a denaturing agent such as formamide, for example, 50% (vol/vol) formamide with 0.1% bovine serum albumin, 0.1% Ficoll, 0.1% polyvinylpyrrolidone, 0.05 M sodium phosphate buffer at pH 6.5 with 0.75 M NaCl, 0.075 M sodium citrate at 42° C. A specific example includes the use of 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 55° C., with washes at 55° C. in 0.2×SSC and 0.1% SDS. A skilled artisan can readily determine and vary the stringency conditions appropriately to obtain a clear and detectable hybridization signal. Preferred molecules are those that hybridize under the above conditions to the complements of Sh2−Rev6 and Sh2-Rev6-HS and which encode a functional protein.

The present invention utilizes nucleic acid molecules encoding SH2−REV6 and SH2-REV6-HS proteins which hybridize with nucleic acid molecules comprising sequences complementary to Sh2−Rev6 and Sh2−Rev6-HS under conditions of sufficient stringency to produce a clear signal. As used herein, “nucleic acid” is defined as RNA or DNA encoding SH2−REV6 and SH2−REV6-HS peptides, or are complementary to nucleic acids encoding such peptides, or hybridize to such nucleic acids and remain stably bound to them under stringent conditions, or encode polypeptides sharing at least 60% sequence identity, or at least 65% sequence identity, or at least 70% sequence identity, or at least 75% sequence identity, or at least 80% sequence identity, or at least 85% sequence identity, preferably at least 90% sequence identity, and more preferably at least 95% sequence identity with the SH2−REV6 and SH2−REV6-HS peptide sequences.

The present invention further provides fragments of any one of the encoding nucleic acids molecules. As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional region(s) of the protein. For instance, fragments of the invention encode the domains or regions of the SH2−REV6 and SH2−REV6-HS of the present invention which are involved with the allosteric regulation of AGP. If the fragment is to be used as a nucleic acid probe or PCR primer, then the fragment length is chosen so as to obtain a relatively small number of false positives during probing and priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention can easily be synthesized by chemical techniques, for example, the phosphotriester method of Matteucci et al. (1981) J. Am. Chem. Soc. 103, 3185-3191) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled encoding nucleic acid molecule.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

III. Isolation of Other Related Nucleic Acid Molecules

As described herein, the identification and characterization of the nucleic acid molecules encoding a SH2−REV6 and SH2−REV6-HS or a fragment of a SH2−REV6 and SH2−REV6-HS allows a skilled artisan to isolate nucleic acid molecules that encode other members of the protein family in addition to the sequences herein described. Further, the presently disclosed nucleic acid molecules allow a skilled artisan to isolate nucleic acid molecules that encode other members of the family of proteins in addition to the SH2−REV6 and SH2−REV6-HS disclosed herein.

Essentially, a skilled artisan can readily use any one of the amino acid sequences disclosed herein to generate antibody probes to screen expression libraries prepared from appropriate cells. Typically, polyclonal antiserum from mammals such as rabbits immunized with the purified protein or monoclonal antibodies can be used to probe a cDNA or genomic expression library to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any organism. Oligomers containing approximately 18-20 nucleotides (encoding about a six to seven amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

IV. Production of Recombinant Proteins Using a Recombinant DNA (rDNA) Molecule

The present invention further provides methods for producing a SH2−REV6 and SH2-REV6-HS protein of the invention using the nucleic acid molecules herein described. In general terms, the production of a recombinant form of a protein typically involves the following steps: First, a nucleic acid molecule is obtained that encodes a SH2−REV6 and SH2−REV6-HS protein or a fragment of a SH2−REV6 and SH2−REV6-HS protein. If the encoding sequence is uninterrupted by introns, it is directly suitable for expression in any host. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences maybe obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host-expression system known in the art for use with the nucleic acid molecules of the invention to produce recombinant protein.

V. SH2−REV6 and SH2−REV6-HS Proteins

As used herein, a SH2−REV6 and SH2−REV6-HS protein refers to a protein that has the amino acid sequence encoded by the polynucleotide of SH2−REV6 and SH2−REV6-HS, allelic variants thereof and conservative substitutions thereof that have SH2−REV6 and SH2−REV6-HS activity. In addition, the polypeptides utilized in the present invention include the proteins encoded by SH2−REV6 and SH2−REV6-HS, as well as polypeptides and fragments, particularly those which have the biological activity of SH2−REV6 and SH2−REV6-HS and also those which have at least 65% sequence identity to the polypeptides encoded by SH2−REV6 and SH2−REV6-HS or the relevant portion, or at least 70% identity, or at least 75% identity, or at least 80% identity, or at least 85% identity to the polypeptides encoded by SH2−REV6 and SH2−REV6-HS or the relevant portion, and more preferably at least 90% similarity to the polypeptides encoded by SH2−REV6 and SH2−REV6-HS or the relevant portion, and still more preferably at least 95% similarity to the polypeptides encoded by SH2−REV6 and SH2−REV6-HS or the relevant portion, and also include portions of such polypeptides. One of skill will recognize whether an amino acid sequence of interest is within a functional domain of a protein, such as the domain or region of the SH2−REV6 and SH2−REV6-HS involved in the allosteric regulation of AGP. Thus, it may be possible for a homologous protein to have less than 40% homology over the length of the amino acid sequence but greater than 90% homology in one functional domain.

The SH2−REV6 and SH2−REV6-HS proteins utilized in the present invention include the specifically identified and characterized variants herein described as well as allelic variants, conservative substitution variants and homologues that can be isolated/generated and characterized without undue experimentation following the methods well known to one skilled in the art.

The term “substantially pure” as used herein refers to SH2−REV6 and SH2−REV6-HS polypeptides which are substantially free of other proteins, lipids, carbohydrates or other materials with which they are naturally associated. One skilled in the art can purify SH2−REV6 and SH2-REV6-HS using standard techniques for protein purification.

The invention also utilizes amino acid sequences coding for isolated SH2−REV6 and SH2-REV6-HS polypeptides. The polypeptides of the invention include those which differ from the exemplified SH2−REV6 and SH2−REV6-HS proteins as a result of conservative variations. The terms “conservative variation” or “conservative substitution” as used herein denotes the replacement of an amino acid residue by another, biologically similar residue. Conservative variations or substitutions are not likely to change the shape of the polypeptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine, and the like. Therefore, all conservative substitutions are included in the invention as long as the SH2−REV6 and SH2−REV6-HS polypeptides encoded by the nucleotide sequence are functionally unchanged or similar.

As used herein, an isolated SH2−REV6 and SH2−REV6-HS protein can be a full-length or any homologue of such SH2−REV6 and SH2−REV6-HS proteins, such as SH2−REV6 and SH2−REV6-HS proteins in which amino acids have been deleted (e.g. a truncated version of the protein, such as a peptide), inserted, inverted, substituted and/or derivatized (e.g., by glycosylation, phosphorylation, acetylation, myristoylation, prenylation, pahnitoylation, amidation and/or addition of glycosylphosphatidyl inositol). Such modified proteins include those that retain at least one of the functional activities of the SH2−REV6 and SH2−REV6-HS proteins or produce at least one of the physiological characteristics produced as a result of the expression of the SH2-REV6 and SH2−REV6-HS proteins. A homologue of a SH2−REV6 and SH2−REV6-HS proteins is a protein having an amino acid sequence that is sufficiently similar to a SH2−REV6 and SH2-REV6-HS protein amino acid sequences that a nucleic acid sequence encoding the homologue is capable of hybridizing under stringent conditions to (i.e., with) a nucleic acid sequence encoding the SH2−REV6 and SH2−REV6-HS protein amino acid sequences. Appropriate stringency requirements are discussed above.

SH2−REV6 and SH2−REV6-HS protein homologues can be the result of allelic variation of a gene encoding a SH2−REV6 and SH2−REV6-HS protein. SH2−REV6 and SH2−REV6-HS protein homologues can be produced using techniques known in the art including, but not limited to, direct modifications to a gene encoding a protein using, for example, classic or recombinant DNA techniques to effect random or targeted mutagenesis.

Minor modifications of the SH2−REV6 and SH2−REV6-HS primary amino acid sequence may result in proteins which have substantially equivalent activity as compared to the SH2−REV6 and SH2−REV6-HS produced by the genes described herein. As used herein, a “functional equivalent” of a SH2−REV6 and SH2−REV6-HS protein is a protein which possesses a biological activity or immunological characteristic substantially similar to a biological activity or immunological characteristic of SH2−REV6 and SH2−REV6-HS. The term “functional equivalent” is intended to include the fragments, variants, analogues, homologues, or chemical derivatives of a molecule which possess the biological activity of the SH2−REV6 and SH2−REV6-HS proteins encoded by the genes of the present invention.

The terms “SH2−REV6 and SH2−REV6-HS proteins,” “SH2−REV6 proteins” and “SH2-REV6-HS proteins” include all allelic variants of these proteins that possess normal SH2−REV6 and SH2−REV6-HS activity. In general, allelic variants of SH2−REV6 and SH2−REV6-HS proteins will have slightly different amino acid sequence than that specifically encoded by the genes utilized in the present invention but will be able to produce the exemplified phenotypes. Allelic variants, though possessing a slightly different amino acid sequence than those recited individual a above, will posses the ability to produce a phenotype which exhibits increased individual and total seed weight, increased seed number, increased Harvest Index (HI) and increased above-ground plant mass.

The methods of the present invention can be used by one skilled in the art to produce plants with increased individual and total seed weight, increased seed number, increased Harvest Index and increased total plant mass.

Applicants further teach methods of recognizing variations in the DNA sequences of Sh2-Rev6 and Sh2−Rev6-HS. One method involves the introduction of a nucleic acid molecule (also known as a probe) having a sequence complementary to the Sh2−Rev6 and Sh2−Rev6-HS genes utilized in the invention under sufficient hybridizing conditions, as would be understood by those in the art. Another method of recognizing DNA sequence variation associated with Sh2−Rev6 and Sh2−Rev6-HS is direct DNA sequence analysis by multiple methods well known in the art. Another embodiment involves the detection of DNA sequence variation in the Sh2−Rev6 and Sh2-Rev6-HS as represented by different plant genera, species, strains, varieties or cultivars. Sh2−Rev6 and Sh2−Rev6-HS can be used as probes to detect the presence of Sh2−Rev6 and Sh2−Rev6-HS genes in other plants. As discussed previously, Sh2−Rev6 and Sh2−Rev6-HS sequences have been determined and are readily available to one of ordinary skill in the art. In one embodiment, the sequences will bind specifically to one allele of a Sh2−Rev6 and Sh2−Rev6-HS gene, or a fragment thereof, and in another embodiment will bind to multiple alleles. Such detection methods include the polymerase chain reaction, restriction fragment length polymorphism (RFLP) analysis and single stranded conformational analysis.

Diagnostic probes useful in such assays of the invention include antibodies to SH2−REV6 and SH2−REV6-HS. The antibodies may be either monoclonal or polyclonal, produced using standard techniques well known in the art (See Harlow & Lane's Antibodies: A Laboratory Manual, Cold Spring Harbor Laboratory Press, 1988). They can be used to detect SH2−REV6 and SH2−REV6-HS proteins by binding to the protein and subsequent detection of the antibody-protein complex by ELISA, Western blot or the like. Antibodies are also produced from peptide sequences of SH2−REV6 and SH2−REV6-HS using standard techniques in the art (See Protocols in Immunology, John Wiley & Sons, 1994). Fragments of the monoclonals or the polyclonal antisera which contain the immunologically significant portion can also be prepared.

Assays to detect or measure SH2−REV6 and SH2−REV6-HS polypeptides in a biological sample with an antibody probe may be based on any available format. For instance, in immunoassays where SH2−REV6 and SH2−REV6-HS polypeptides are the analyte, the test sample, typically a biological sample, is incubated with anti-SH2−REV6 and SH2−REV6-HS antibodies under conditions that allow the formation of antigen-antibody complexes. Various formats can be employed, such as “sandwich” assay where antibody bound to a solid support is incubated with the test sample; washed, incubated with a second, labeled antibody to the analyte; and the support is washed again. Analyte is detected by determining if the second antibody is bound to the support. In a competitive format, which can be either heterogeneous or homogeneous, a test sample is usually incubated with an antibody and a labeled competing antigen, either sequentially or simultaneously. These and other formats are well known in the art.

VI. Transformation Methods

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation (see, e.g., U.S. Pat. Nos. 5,405,765, 5,472,869, 5,538,877, 5,538,880, 5,550,318, 5,641,664, 5,736,369 and 5,736369; Watson et al. (1992) Recombinant DNA, Scientific American Books; Hinchee et al. (1988) Bio/Tech 6:915-922 (1988); McCabe et al, Bio/Tech. 6:923-926; Toriyama et al. (1988) Bio/Tech 6:1072-1074; Fromm et al. (1990) Bio/Tech 8:833-839; Mullins et al. (1990) Bio/Tech. 8:833-839; and, Raineri et al. (1990) Bio/Tech. 8:33-38).

A. Agrobacterium-Mediated Transformation

Agrobacterium-mediated transformation is the most widely utilized method for introducing an expression vector into plants (Horsch et al. (1985) Science 227:1229). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. (Kado, C. I. (1991) Crit. Rev. Plant. Sci. 10:1). Descriptions of Agrobacterium vector systems and methods for Agrobacterium-mediated gene transfer are provided by Gruber et al. (1993) “Vectors for Plant Transformation” in Methods in Plant Molecular Biology and Biotechnology, Glick, B. R. and Thompson, J. E. Eds. (CRC Press, Inc., Boca Raton), pages 89-119), Miki et al. (1993) “Procedures for Introducing Foreign DNA into Plants” in Methods in Plant Molecular Biology and Biotechnology, Glick, B. R. and Thompson, J. E. Eds. (CRC Press, Inc., Boca Raton) pages 67-88, and Moloney et al. (1989)

Plant Cell Reports 8:238.

Agrobacterium-mediated transformation methods have been used principally to transform dicotyledonous plants. Agrobacterium-mediated transformation in dicotyledons facilitates the delivery of larger pieces of heterologous nucleic acid as compared with other transformation methods such as particle bombardment, electroporation, and polyethylene glycol-mediated transformation method. In addition, Agrobacterium-mediated transformation appears to result in relatively few gene rearrangements and more typically results in the integration of low numbers of gene copies into the plant chromosome.

Monocotyledons are not a natural host of Agrobacterium. Although Agrobacterium-mediated transformation has been reported for asparagus (Bytebier et al (1987) Proc. Natl. Acad. Sci. USA 84:5354-5349) and for Dioscore bublifera (Schafer et al (1987) Nature 327:529-532), it was generally believed that plants in the family Gramineae could not be transformed with Agrobacterium (Potrykus I. (1987) Biotechnology 8:535-543). However, recently in U.S. Pat. No. 5,981,840, Zhao et al. disclosed agrobacteriun-mediated transformation in maize. The method of Zhao et al. includes the following steps: contacting at least one immature embryo from a maize plant with Agrobacterium capable of transferring at least one gene to said embryo; co-cultivating the embryos with Agrobacterium; culturing the embryos in medium comprising N6 salts, an antibiotic capable of inhibiting the growth of Agrobacterium, and a selective agent to select for embryos expressing the gene; and regenerating plants expressing the gene.

B. Microprojectile-Mediated Transformation

In a microprojectile bombardment process, also referred to as a biolistic process, the transport of the DNA is mediated by very small particles of a biologically inert material. When the inert particles are coated with DNA and accelerated to a suitable velocity, one or more of the particles is able to enter into one or more of the cells where the DNA is released from the particle and expressed within the cell. While some of the cells are fatally damaged by the bombardment process, some of the recipient cells do survive, stably retain the introduced DNA, and express it. Sanford et al. provides a general description of a suitable particle bombardment instrument (Sanford et al. (1987) Particulate Sci. Technol. 5: 27-37).

Microprojectile bombardment process has been used to successfully introduce genes encoding new genetic traits into a number of plants, including onion, cotton, maize, tobacco, rice, wheat, sunflowers, soybeans and certain vegetables (U.S. Pat. No. 4,945,050; Sanford et al. (1988) Trends in Biotechnology 6:299; Sanford et a. (1988) Part. Sci. Technol. 5:27; J. J. Finer and M. D. McMullen (1990) Plant Cell Reports 8:586-589; and Gordon-Kamm (1990) The Plant Cell 2:603; Klein et al. (1988) Proc. Nat. Acad. Sci. USA 85:4305-4309). Although transformation by microprojectile bombardment is less species and genotype specific than transformation with Agrobacterium, the frequencies of stable transformation events achieved following bombardment can be quite low, partly due to the absence of a natural mechanism for mediating the integration of a DNA molecule or gene responsible for a desired phenotypic trait into the genomic DNA of a plant. Particle gun transformation of cotton for example, has been reported to produce no more than one clonal transgenic plant per 100-500 meristems targeted for transformation. Only 0.1 to 1% of these transformants were capable of transmitting foreign DNA to progeny (WO 92/15675). Cells treated by particle bombardment must be regenerated into whole plants, which requires labor intensive, sterile tissue culture procedures and is generally genotype dependent in most crop plants, particularly so in cotton. Similar low transformation frequencies have been reported for other plant species as well. Other disadvantages of microprojectile bombardment include the inability to control the site of wounding of a plant tissue and thus, the site to which the transforming agent is delivered. The inability to target germline tissues accounts in part for the low transformation efficiencies achieved by microprojectile bombardment. Additionally, bombardment frequently results in the delivery of more than one copy of the transforming DNA or gene into the genome of the transformed plant cell, which can have deleterious effects on the regenerated, transformed plant. Fragmentation of the DNA to be inserted can also occur when transforming DNA via microprojectile bombardment, resulting in transgenic plants with only a portion of the gene that is being inserted.

Attempts have been made to improve the efficiency of microprojectile bombardment. For example, EPA 0486 233 describes treating bombarded tissues with Agrobacterium carrying the gene of interest. It is thought that the high velocity impact of the dense microprojectile particles generates an array of microwounds creating an environment that is particularly conducive to infection by the Agrobacterium. However, the transformed plant cells must still be regenerated into whole plants, and the fertile, stably transformed plants must be selected from the total population of regenerated plants. Organogenesis and somatic embryogenesis have been used to regenerate plants. Nonetheless, organogenesis frequently produces chimeric plant containing both transformed and nontransformed cells, and somatic embryogenesis, although superior to organogenesis is highly genotype dependent in most crop plants.

Efforts have been made to deliver the transforming agent or DNA to germline tissues such that the agent or DNA will be incorporated directly into the DNA of the cells in these tissues, particularly into the DNA of the egg cells of the plant. In U.S. Pat. No. 5,994,624, Trolinder et al. describes a method of implanta transformation which provides an improved method for delivering transforming agents to plant tissues. The method uses a needleless-injection device that is capable of injecting a small high pressure stream of a solution through the many cell layers of plant tissue. The transforming agent is delivered to a plant's floral tissues, thereby facilitating delivery of a transforming agent comprising a gene of interest into germline cells of the plant. The high pressure stream provided by the injection device insures that the Agrobacterium culture or the DNA solution penetrates the many cell layers of the plant floral tissue without causing massive tissue damage, such as that caused by direct injection with a syringe having a needle or by particle bombardment. The method can be used to transform plant cells and tissues, including embryonic tissue culture cells, meristematic tissues and plant callus, which can be regenerated into whole plants. Moreover, the method can be used to transform plant cells and tissues selected from the group consisting of cotton, soybean, alfalfa, flax, tobacco, sunflower, peanut, strawberry, tomato, pea, bean, squash, pepper, maize, sorghum, barley, oat, rye, wheat, rice, brassica and potato.

Although Klein et al. (Klein et al. (1988) Proc. Nat. Acad. Sci. USA 85:4305-4309; Klein et al. (1988) Bio/Technol. 6:59-563; Klein et al. (1989) Plant Physiol. 91:440-444) provides protocols for bombardment of maize non-regenerable suspension culture cells, no protocols have been published for the bombardment of callus cultures or regenerable maize cells until recently. Lundquist et al. (U.S. Pat. No. 6,013,863) describes delivery of DNA into regenerable maize callus cultures via particle bombardment process which results in high level of viability for a few transformed cells. The method maybe applicable to producing fertile stably transgenic plants of other graminaceous cereals. Dwight et al. (U.S. Pat. No. 5,990,387) discloses a method of producing fertile, stably transformed, Zea mays plant. The methods comprise the following steps: providing a foreign DNA comprising an expression vector carrying a gene encoding an agronomic trait; providing a maize embryogenic callus, suspension culture, or immature embryo isolated from a plant; introducing the foreign DNA into the embryogenic callus, suspension culture or immature embryo isolated from a plant by one or more microparticle bombardments; and regenerating fertile transgenic Zea mays plant. Plants that can be successfully transformed by the method of Dwight et al. include maize, rye, barley, wheat, sorghum, oats, millet, rice, sunflower, alfalfa, rape seed and soybean.

Biswas et al. describes generation of transgenic rice plants by microprojectile bombardment of embryogenic cell clusters (Biswas et al. (1998) Plant Science, 133:203-210), and Yao et al. discloses the production of transgenic barley plants via direct delivery of plasmid DNA into microspores of barley using high velocity microprojectiles (Yao et al. (1997) Genome, 40:570-581). Christou et al. reports on the parameters that influence stable transformation of rice embryogenic callus and the recovery of transgenic plants using electric discharge particle acceleration (Christou et al. (1995) Annals of Botany 75:407-413).

C. Alternative Methods of Transformation

Other methods for physical delivery of DNA to plants include sonication of target cells (Zhang et al. (1991) Bio/Technology 9:996) and liposome or spheroplast fusion (Deshayes et al. (1985) EMBO J., 4: 2731, Christou et al. (1987) Proc Natl. Acad. Sci. USA 84: 3962). Direct uptake of DNA into protoplasts using CaCl₂ precipitation, polyvinyl alcohol or poly-L-omithine has also been reported (Hain et al. (1985) Mol. Gen. Genet. 199: 161 and Draper et al. (1982) Plant Cell Physiol. 23: 451). Nobre et al. reports the regeneration of fertile transgenic plants of barley using PEG-mediated transformation of scutellum protoplast (Nobre et al. (1997) Barley Genetics Newsletter, 27:16-17). Electroporation of protoplasts and whole cells and tissues has also been described (Donn et al. (1990) Abstracts of International Congress on Plant Cell and Tissue Culture IAPTC, A2-38, p 53; D'Halluin et al. (1992) Plant Cell 4: 1495-1505 and Spencer et al. (1994) Plant Mol. Biol. 24: 51-61). In fact, D'Halluin et al. (U.S. Pat. No. 6,002,070) describes a rapid and efficient method of transforming monocotyledonous plants by electroporation. The method of D'Halluin comprises electroporation of DNA of interest into either intact tissue capable of forming compact embryogenic callus or compact embryogenic callus obtained from intact tissue.

Another technology for production of transgenic plants is whisker-mediated transformation whereby certain materials, when incubated with plant tissue, facilitate entry of DNA molecules into plant cells. It has been proposed that such materials that promote DNA uptake, primarily silicone carbide, do so by damaging the cell surface. For a review, see Wang et al. (1995) In Vitro Cell. Dev. Biol. 34: 101-4.

VII. Transgenes

Genes successfully introduced into plants using recombinant DNA methodologies include, but are not limited to, those coding for the following traits: seed storage proteins, including modified 7S legume seed storage proteins (U.S. Pat. Nos. 5,508,468, 5,559,223 and 5,576,203); herbicide tolerance or resistance (U.S. Pat. Nos. 5,498,544 and 5,554,798; Powell et al. (1986) Science 232:738-743; Kaniewski et al. (1990) Bio/Tech. 8:750-754; Day et al. (1991) Proc. Natl. Acad. Sci. USA 88:6721-6725); phytase (U.S. Pat. No. 5,593,963); resistance to bacterial, fungal, nematode and insect pests, including resistance to the lepidoptera insects conferred by the Bt gene (U.S. Pat. Nos. 5,597,945 and 5,597,946; Hilder et al. Nature 330:160-163; Johnson et al. (1989) Proc. Natl. Acad. Sci. USA 86:9871-9875; Perlak et al. (1990) Bio/Tech. 8:939-943); lectins (U.S. Pat. No. 5,276,269); and flower color Meyer et al. (1987) Nature 330:677-678; Napoli et al.(1990) Plant Cell 2:279-289 (1990); van der Krol et al. (1990) Plant Cell 2:291-299).

VIII. Expression Units to Express Exogenous DNA in a Plant

The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include any plant species.

Any prokaryotic host can be used to express a rDNA molecule encoding a protein of the invention. The preferred prokaryotic host is E. coli. Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al. (1972) Proc. Natl. Acad. Sci. USA 69:2110-2114; and Maniatis et al. (1982) Molecular Cloning—A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al. (1973) Virology 52:456-467; and Wigler et al. (1979) Proc. Natl. Acad. Sci. USA 76:1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, (1975) J. Mol. Biol. 98:503-517; or Berent et al. (1985) Biotech. Histochem. 3:208; or the proteins produced from the cell assayed via an immunological method.

As provided herein elsewhere, several embodiments of the present invention employ expression units (or expression vectors or systems) to express an exogenously supplied nucleic acid sequence, such as the sequence coding for SH2−REV6 and SH2−REV6-HS protein in a plant. Methods for generating expression units/systems/vectors for use in plants are well known in the art and can readily be adapted for use in expressing the SH2−REV6 and SH2−REV6-HS proteins in a plant cell. A skilled artisan can readily use any appropriate plant/vector/expression system in the present methods following the outline provided herein.

The expression control elements used to regulate the expression of the protein can either be the expression control element that is normally found associated with the coding sequence (homologous expression element) or can be a heterologous expression control element. A variety of homologous and heterologous expression control elements are known in the art and can readily be used to make expression units for use in the present invention. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 35S promoter to control gene expression in a plant. Lastly, plant promoters such as prolifera promoter, fruit-specific promoters, Ap3 promoter, heat shock promoters, seed-specific promoters, etc. can also be used. The most preferred promoters will be most active in seedlings.

Either a constitutive promoter (such as the CaMV or Nos promoter), an organ-specific promoter (such as the E8 promoter from tomato) or an inducible promoter is typically ligated to the protein or antisense encoding region using standard techniques known in the art. The expression unit may be further optimized by employing supplemental elements such as transcription terminators and/or enhancer elements.

Thus, for expression in plants, the expression units will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a preexisting vector.

In the construction of heterologous promoter/structural gene or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. Polyadenylation sequences include, but are not limited to the Agrobacterium octopine synthase signal (Gielen et al. (1984) EMBO J 3:835-846) or the nopaline synthase signal (Depicker et al. (1982) Mol. and Appl. Genet. 1: 561-573).

The resulting expression unit is ligated into or otherwise constructed to be included in a vector which is appropriate for higher plant transformation. The vector will also typically contain a selectable marker gene by which transformed plant cells can be identified in culture. Usually, the marker gene will encode antibiotic resistance. These markers include resistance to G418, hygromycin, bleomycin, kanamycin, and gentamicin. After transforming the plant cells, those cells having the vector will be identified by their ability to grow on a medium containing the particular antibiotic. Replication sequences, of bacterial or viral origin, are generally also included to allow the vector to be cloned in a bacterial or phage host, preferably a broad host range prokaryotic origin of replication is included. A selectable marker for bacteria should also be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

The Sh2−Rev6 and Sh2−Rev6-HS sequences utilized in the present invention can also be fused to various other nucleic acid molecules such as Expressed Sequence Tags (ESTs), epitopes or fluorescent protein markers.

ESTs are gene fragments, typically 300 to 400 nucleotides in length, sequenced from the 3′ or 5′ end of complementary-DNA (cDNA) clones. Nearly 30,000 Arabidopsis thaliana ESTs have been produced by a French and an American consortium (Delseny et al. (1997) FEBS Lett. 405(2):129-132; Arabidopsis thaliana Database, http://genome.www.stanford.edu/Arabidopsis). For a discussion of the analysis of gene-expression patterns derived from large EST databases, see, e.g., M. R. Fannon (1996) TIBTECH 14:294-298.

Biologically compatible fluorescent protein probes, particularly the self-assembling green fluorescent protein (GFP) from the jellyfish Aequorea victoria, have revolutionized research in cell, molecular and developmental biology because they allow visualization of biochemical events in living cells (Murphy et al. (1997) Curr. Biol. 7(11):870-876; Grebenok et al. (1997) Plant J. 11(3):573-586; Pang et al. (1996) Plant Physiol. 112(3); Chiu et al. (1996) Curr. Biol. 6(3):325-330; Plautz et al., (1996) Gene 173(1):83-87; Sheen et al. (1995) Plant J. 8(5):777-784).

Site-directed mutatgenesis has been used to develop a more soluble version of the codon-modified GFP call soluble-modified GFP (smGFP). When introduced into Arabidopsis, greater fluorescence was observed when compared to the codon-modified GFP, implying that smGFP is ‘brighter’ because more of it is present in a soluble and functional form (Davis et al. (1998) Plant Mol. Biol. 36(4):521-528). By fusing genes encoding GFP and beta-glucuronidase (GUS), researchers were able to create a set of bifunctional reporter constructs which are optimized for use in transient and stable expression systems in plants, including Arabidopsis (Quaedvlieg et al. (1998) Plant Mol. Biol. 37(4):715-727).

Berger et al. (Berg et al. (1998) Dev. Biol. 194(2):226-234) report the isolation of a GFP marker line for Arabidopsis hypocotyl epidermal cells. GFP-fusion proteins have been used to localize and characterize a number of Arabidopsis genes, including geranylgeranyl pyrophosphate (GGPP) (Zhu et al. (1997) Plant Mol. Biol. 35(3):331-341).

IX. Breeding Methods

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population which is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, (1960) Principles of Plant Breeding, John Wiley & Sons, Inc.; Simmonds (1979) Principles of Crop Improvement, Longman Group Limited; Hallauer and Miranda (1981) Quantitative Genetics in Maize Breeding, Iowa State University Press; and, Jensen (1988) Plant Breeding Methodology, John Wiley & Sons, Inc.

Mass Selection. In mass selection, desirable individual plants are chosen, harvested, and the seed composited without progeny testing to produce the following generation. Since selection is based on the maternal parent only, and their is no control over pollination, mass selection amounts to a form of random mating with selection. As stated above, the purpose of mass selection is to increase the proportion of superior genotypes in the population.

Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g., by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Hybrids. A hybrid is an individual plant resulting from a cross between parents of differing genotypes. Commercial hybrids are now used extensively in many crops, including corn (maize), sorghum, sugarbeet, sunflower and broccoli. Hybrids can also be produced in wheat and rice. Hybrids can be formed a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an outbreeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity which results in increased vigor of growth, survival, and fertility of hybrids as compared with the parental lines which were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Corp Plants, supra.

X. Seed Number, Grain Yield, and Sink Capacity in Wheat

Wheat seed number and subsequent grain yield is affected by competition between inflorescences (Whingwiri et al., 1981). Wheat yield is always lower than ears potential due to lack of assimilate supply or competition among florets limiting seed size and/or number (Zamski and Grunberger, 1995). Healthy, well-grown wheat plants always produce more shoots (potential heads) and florets (potential seeds) than heads and seeds. A significant factor controlling seed number is sink strength of the developing seeds (Thorne and Wood, 1987). A review of this area (Evans et al., 1975) indicates for wheat in many cases, yield is limited by the sink capacity of developing seeds. The limitations imparted by low sink strength may be seen as reduced grain set, reduced number of wheat heads, and reduced individual seed weight. In wheat, it is generally believed that the rate of assimilate flow to developing heads determines the survival of initiated florets and plays a significant role in determining final grain number (Spiertz and vanKeulen, 1980; Abbate et al., 1998). Possibly the most effective method of increasing kernel number in wheat would be to modify assimilate flow to developing kernels (Bindraban et al., 1998). The transgenic wheat of the present invention which contains increased sink strength confirms this hypothesis.

### Materials and Methods

I. Production of Transgenic Plants

Vectors according to the invention may be used to transform plants as desired, to make plants according to the invention as discussed elsewhere herein.

Wheat Transformation. The methods described by Weeks et al. (1993) and Vasil et al. (1993) have been adopted with minor modifications for transforming the wheat cultivar ‘Hi-Line’ (Lanning et al., 1992). The technique as routinely practiced initially utilizes immature embryos isolated from wheat cultivars approximately 7 days post anthesis.

The Biolistic PDS-1000 He (Bio-Rad laboratories, USA) device was used for transforming the wheat tissues via microprojectile bombardment.

For wheat calli 1500 psi rupture discs were used. Other procedures such as sterilization of the rupture discs, macrocarriers, stopping screens etc., were strictly in accordance with the manufacturer's manual.

Rice Transformation. The methods described by Sivamani et al. (1996) may be adopted for transforming rice cultivar ‘M202’ (Johnson et al. 1986). The technique as routinely practiced initially utilizes embryogenic calli cultured from mature seeds.

The Biolistic PDS-1000 He (Bio-Rad laboratories, USA) device is used for transforming the rice tissues via microprojectile bombardment.

For rice calli 1500 psi rupture discs are used. Other procedures such as sterilization of the rupture discs, macrocarriers, stopping screens etc., are strictly in accordance with the manufacturer's manual.

Pea Transformation. The methods described by U.S. Pat. No. 5,286,635 (Example 9) and U.S. Pat. No. 5,773,693 (Example V) may be adopted with minor modifications for transforming the pea (Pisum sativum L.) cultivar ‘Pea Green Arrow’ (available commercially from Park Seed®). Pea explant material is transformed by incubation with Agrobacterium cells carrying Sh2−Rev6-HS sequence. The pea explant is preferably obtained from the plumule of a pea seed, and transformed shoots are preferably induced directly in the explant material without passage through a callus phase. Whole transformed pea plants may be regenerated from the transformed shoots by rooting and subsequent planting in the soil. The exogenous Sh2−Rev6-HS DNA will be stably incorporated into the chromosomes of the regenerated ‘Pea Green Arrow’ plant which will be able to express the gene.

II. Plasmids

Wheat. The plasmid DNA pRQ101 containing the coding sequence of the Bar gene (Fromm et al, 1990) under control of the CaMV 35S promotor with AdhI intron and NOS terminator was used as selectable marker for selecting transgenic wheat tissue.

Rice. As a selectable marker for rice, the plasmid DNA pILTAB222 containing the coding sequence of the hygromycin B phosphotransferase under the control of the maize ubiquitin promoter was used (Sivamani et al., 1996).

Pea. As a selectable marker for pea, the coding sequence of cefotoxime resistance may be used according to U.S. Pat. No. 5,773,693. This anti-Agrobacterium antibiotic may be used in the selection and regeneration medium (500 mg/l) used for growing the pea callus.

General. The marker genes (i.e., Bar, hygromycin resistance, or cefotaxime) were on different construct than Sh2−Rev6-HS genes.

For the introduction of the Sh2−Rev6-HS genes into cereals, plasmid pSh2−Rev6-HS was created. Besides containing Sh2−Rev6-HS cDNA, the plasmid also contained the Sh2 promoter, Sh1 first intron, and NOS terminator (Rogers et al., 1987). Specifically, plasmid pSh2−Rev6-HS contains the following nucleotide fragments linked in the 5′ to 3′ direction: nucleotides −1084 to +36 of the Sh2 promoter; 8 nucleotides of polylinker; two C's; nucleotides of the Sh1 intron 1 cassette containing nucleotides +43 to +52 of Sh1 exon 1, nucleotides +53 to +1080 of Sh1 intron 1 and nucleotides +1081 to 1097 of Sh1 exon 2; one C; 13 nucleotides of polylinker containing a BamH1 restriction site; cDNA encoding Sh2−Rev6-HS (SEQ ID NO: 3); 18 nucleotides polylinker containing KpnI and SstI restriction sites; and nucleic acid of the NOS terminator. The nucleic acid sequence of the Sh2 promoter is disclosed by Shaw and Hannah, (1992), Plant Physiology, 98:1214-1216. The sequence numbering of Sh1 intron cassette is shown in Zack et al. (1986) Maydica, 31, 5-16, and the effects of the Sh1 intron1 cassette on transient gene expression are described by Clancy et al. ((1994) Plant Science, 98, 151-161) and Vasil et al. ((1989), Plant Science, 91, 1575-1579). The 3 additional C's (2 at the 5′ end and 1 at the 3′ end) are subcloning derived nucleotides. The plasmid includes transit peptide and consensus start site. Plasmid pSh2−Rev6-HS used in the present application is provided by Florida State University.

For introduction of Sh2−Rev-HS into dicots such as pea, the above plasmid is modified such that the Sh2 promoter is replaced with a dicot seed specific promoter such as pea vicilin promoter (U.S. Pat. No. 5, 773, 693). Other suitable promoters and/or constructs for expression of Sh2−Rev6-HS in dicots are well known to the skilled artisan (see, e.g., U.S. Pat. No. 5,773,693).

III. Selection and Regeneration of Transgenic Plants

Wheat. Transgenic wheat plants were obtained from bombarded immature embryos by the methods described by Weeks et al. (1993) and Vasil et al. (1993) using bialaphos (Meiji Seika Kaisha Ltd, Japan) selection. The resistant calli of wheat are transferred to medium to induce production of both shoots and roots.

Rice. Transgenic rice plants were obtained from the bombarded embryogenic calli of rice by the technique of Sivamani et al. (1996) using hygromycin selection. The resistant calli of rice are transferred to medium to induce production of both shoots and roots.

Peas. Transgenic pea plants maybe obtained from Agrobacterium-transformed calli of pea explants by the method of U.S. Pat. No. 5,773,693 using cefotaxime selection.

Pea shoots may be rooted by transfer to Sorbarod plugs (Baumgartnen Papiers SA, Switzerland) and soaked in liquid YRM according to U.S. Pat. No. 5,773,693 (Example V).

General. Putative transgenic plantlets were transferred to the greenhouse and allowed to self-fertilize. For wheat, typically more than 75% of these plantlets are escapes and true transgenic plants were selected by spraying the plants with 0.1% glufosinate (Liberty®, Agrevo Inc.).

IV. Primers for PCR

We used a Sh2 specific primer and a NOS specific primer for PCR to confirm the presence of Sh2−Rev6-HS transgene in the transgenic plants. The 5′primer was MC4Sh2, a 26-mer which is specific to Sh2 sequences in the construct:

5′ CTG GAT GTG AAC TCA AGG ACT CCG TG 3′ (SEQ ID NO: 5).

The 3′ primer was MC35PUC19, a 24-mer specific to the puc backbone of the construct:

5′ GGC TTA ACT ATG CGG CAT CAG AGC 3′ (SEQ ID NO: 6).

The primers produce a PCR product of 826 bp (309 of Sh2 cDNA, 260 bp of NOS, and 257 bp pUC19).

### EXAMPLE 1

### Genetic Analysis of Transgenic Wheat Plants

The initial pool of wheat transformants yielded a number of independent transformants which were transgenic for Sh2−Rev6-HS and/or basta resistance.

The T₀ plants were allowed to set seed and mature in the greenhouse under controlled conditions.

The selected wheat transformants were analyzed by PCR for the presence of the introduced transgene and for T₁ seed segregation data for basta resistance.

PCR screening of transgenic wheat plants utilized MC4Sh2 and MC35PUC19 (primer sequences given above) for the presence of Sh2−Rev6-HS in genomic DNA samples prepared from leaf tissue using standard PCR protocols.

Twenty seven independent lines of transgenic wheat were tested. All 27 transgenic lines tested positive for basta resistance. Fifteen of the 27 transgenic lines tested positive for the presence of the Sh2−Rev6-HS transgene and the other 12 did not test positive for the presence of the Sh2−Rev6-HS transgene.

### EXAMPLE 2

### Phenotypic Analysis of the Transgenic Wheat Plants

Various phenotypic traits were collected and analyzed for each of the 27 transgenic wheat plants grown in a greenhouse. As mentioned previously, all 27 transgenic lines carried the herbicide resistance gene. These traits included the following: number of seeds per plant (Seeds/Plant); individual seed weight (Individual Seed Wt.) in milligrams per kernel (mg/kernel); Harvest Index (Harvest Index); total seed weight (Total Seed Wt.) in grams per plant (g/plant); number of grain heads per plant (Heads); total plant weight (Plant Wt.) in grams per plant (g/plant); and flag leaf weight (Flag Leaf Wt.) in grams per plant (g/plant).

Seeds were uniformly dried in a 37° C. incubator to a moisture of between about 10% to about 14%.

The above ground parts of the plants were harvested at time of maturity and uniformly dried to about 0% moisture in a 125° C. incubator. The dried plant weights and dried flag leaf weights were adjusted to reflect weights at the same moisture content as that of the seeds (i.e., about 10% to about 14%). Roots were not collected.

Plant weight represents the total weight of the “above ground” plant parts not including the total seed weight of the plant and the flag leaf weight of the plant.

Harvest Index (HI) was calculated as follows:

HI={Total Seed Wt.)/(Total Seed Wt.+Plant Wt.+Flag Leaf Wt.)}.

For the number of wheat heads per plant, the number of heads were counted without regard to whether or how many seeds were in any particular head.

The phenotypic data were analyzed in several different ways, as discussed below.

Comparison Between PCR+ and PCR− Lines. This comparison was made for all transgenic lines (15 lines) with positive PCR results (PCR+) for Sh2−Rev6-HS versus all transgenic lines (12 lines) with negative PCR results (PCR−) for Sh2−Rev6-HS. Thus, the PCR+ lines carry both the herbicide resistance gene and the Sh2−Rev6-HS gene while the PCR− lines only carry the herbicide resistance genes. The results are presented in Table I.

TABLE I
Comparison Between PCR+ and PCR− Lines

| | | | Individual | Total | | | | Flag Leaf | |
|---|---|---|---|---|---|---|---|---|---|
| Number and Types | | Seeds/ | Seed Wt. | Seed Wt. | Harvest | Heads | Plant Wt. | Wt. | # of |
| of Lines | | Plants | (mg/kernel) | (g/plant) | Index | # | (g/plant) | (g/plant) | Plants |
| 15 PCR+ | Avg | 63.68** | 25.2*** | 1.73** | 0.30** | 5.71 | 3.74 | 0.40 | 183 |
| | Std | 10.59 | 1.8 | 0.34 | 0.08 | 0.86 | 2.38 | 0.20 | |
| 12 PCR− | Avg | 53.50 | 24.10 | 1.37 | 0.25 | 5.82 | 3.64 | 0.41 | 148 |
| | Std | 9.60 | 1.50 | 0.26 | 0.10 | 0.81 | 1.74 | 0.18 | |
| PCT+/PCR− | | 1.19* | 1.05*** | 1.26** | 1.17** | 0.98 | 1.03 | 0.97 | |

*, **, ***indicate p values of less than or equal to: 0.05, 0.01, or 0.001, respectively, based on a t test.

Comparison Between SH2+ and SH2−. In the second comparison, only the 8 transgenic lines with positive PCR results for Sh2−Rev6-HS which also displayed increases in the levels of the introduced protein (SH2+) were averaged and compared with all other lines (SH2−). The 8 PCR+ lines which are SH2+ are the lines for which increased levels of the introduced protein were detected. Basically, the SH2 levels were compared to those of the lines which were transgenic for only the herbicide resistance gene. Those experimental plants which produced 25% or more of the SH2 protein as compared to the production of SH2 by the lines which were transgenic for only the herbicide resistance gene were designated as “SH2+”.

The SH2+ lines were compared to the other 19 lines (“SH2−”) which lack any significant expression of the introduced protein. Thus, the 19 SH2−lines includes the 7 PCR+ lines which did not express significant levels of the SH2−REV6-HS protein and the 12 PCR− lines which did not express SH2−REV6-HS at all. The data is presented in Table II.

TABLE II
Comparison Between SH2+ and SH−

| | | | Individual | Total Seed | | | | Flag Leaf | |
|---|---|---|---|---|---|---|---|---|---|
| Number and | | Seeds/ | Seed Wt. | Wt. | Harvest | | Plant Wt. | Wt. | # of |
| Types of Lines | | Plants | (mg/kernel) | (g/plant) | Index | Heads | (g/plant) | (g/plant) | Plants |
| 8 SH2+ | Avg | 78.23*** | 26.9*** | 2.19** | 0.32*** | 6.00 | 4.29*** | 0.45** | 100 |
| | Std | 45.50 | 4.10 | 1.35 | 0.10 | 2.93 | 2.29 | 0.21 | |
| 19 SH2− | Avg | 50.86 | 23.80 | 1.30 | 0.25 | 5.66 | 3.44 | 0.39 | 231 |
| | Std | 33.65 | 4.70 | 0.94 | 0.13 | 2.38 | 1.99 | 0.18 | |
| SH2+/SH2− | | 1.54*** | 1.13*** | 1.68*** | 1.25*** | 1.06 | 1.25*** | 1.15** | |

*, **, ***indicate p values of less than or equal to: 0.05, 0.01, or 0.001, respectively, based on a t-tests.

The data presented in Table II show that the total number of seeds per plant for the SH12+ lines increased about 54% in comparison to the total number of seeds per plant for the SH2− lines. Individual seed weight increased about 13% and the total seed yield increased about 68% for the SH12+ lines when compared to the SH2− lines. Harvest Index for the SH+ lines was about 25% greater than that for the SH2− lines. The SH2+ lines were also significantly bigger in total plant mass and in flag leaf weight (about +25% and about +15%, respectively).

Comparison Between Lines Homozygous and Heterozygous for Sh2−Rev6-HS. T1 plants found to be homozygous by progeny testing the T2 seeds were designated as “Homoz SH2+”. The seeds of Homoz SH2+ plants are expected to have a greater dosage of the transgene than the other lines. In this comparison, the Homoz SH2 plants were compared to SH2+ plants that were heterozygous (Heteroz SH2+) and were also compared to the 12 PCR− lines.

TABLE III
Comparison Between Lines Homozygous and Heterozygous for Sh2-Rev6-HS

| | | | | | | | | |
|---|---|---|---|---|---|---|---|---|
| | | Individual | Total Seed | | | Plant | Flag Leaf | |
| Types of | Seeds/ | Seed Wt. | Wt. | Harvest | | Wt. | Wt. | # of |
| Lines | Plants | (mg/kernel) | (g/plant) | Index | Heads | (g/plant) | (g/plant) | Plants |

| | | | | | | | | | |
|---|---|---|---|---|---|---|---|---|---|
| Homoz | Avg | 97.1* | 27.60 | 2.74* | 0.30 | 7.59** | 5.76 | 0.56 | 22 |
| SH2+ | Std | 43.60 | 2.60 | 1.31 | 0.03 | 3.10 | 2.25 | 0.17 | |
| Heteroz | Avg | 74.50 | 26.80 | 2.10 | 0.33 | 5.58 | 3.91 | 0.42 | 66 |
| SH2+ | Std | 45.10 | 3.80 | 1.34 | 0.10 | 2.54 | 2.08 | 0.20 | |
| SH2+ Homoz/ | | 1.30* | 1.03 | 1.30* | −0.05 | 1.36** | 1.47 | 1.33 | 22/66 |
| Heteroz | | | | | | | | | |
| SH2 Homoz/ | | 1.91*** | 1.16*** | 2.10*** | 1.16* | 1.34** | 1.67*** | 1.43*** | 22/148 |
| 12 PCR− | | | | | | | | | |

*, **, ***indicate p values of less than or equal to: 0.05, 0.01, or 0.001, respectively, based on t tests.

The majority (approximately ⅔'s) of the plants analyzed were determined to be heterozygous for Sh2−Rev6-HS and therefore only have ¹2 of the possible dosage of the transgene coding for SH2−REV6-HS.

To determine the effect of increased gene dosage, individual T₁ plants were determined to be homozygous or heterozygous by progeny testing the T₂ seeds harvested from the plants. Lack of segregation for the herbicide resistance marker gene was taken as evidence of homozygosity. A comparison of 22 SH2+ homozygous plants with heterozygous SH+ plants indicates that increasing the dosage of the Sh2−Rev6-HS transgene leads to even larger yield and plant growth increases over plants which do not contain or express the transgene. The results provided in Table III indicate an approximately 110% increase in total seed weight per plant compared to SH2+ heterozygous plants.

### EXAMPLE 3

### Experiments With Rice

The transgenic rice plants are produced as described in the Materials and Methods. The resultant rice plants are analyzed as set forth in Examples 1 and 2.

### EXAMPLE 4

### Experiments With Pea

The transgenic pea plants are produced as described in the Materials and Methods. The resultant pea plants are analyzed as set forth in Examples 1 and 2.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES FOR WHICH A COMPLETE CITATION IS NOT PROVIDED IN THE TEXT OF THE SPECIFICATION.

Bae, J. M., Giroux, M. Hannah, L. (1990) Cloning and characterization of the brittle-2 gene of maize. Maydica 35:317-322.

Bhave, M. R., Lawrence, S., Barton, C., and Hannah, L. C. (1990) Identification and molecular characterization of shrunken-2 cDNA clones of maize. Plant Cell 2:581-588.

Choi, S-B., Zhang, Y., Ito, H., Stephens, K., Winder, T., Edwards, G. E., and Okita, T. W. (1998) Increasing rice productivity by manipulation of starch biosynthesis during seed development. p. 137-149 In Feeding a world population of more than eight billion people: a challenge to science. Ed. J. C. Waterlow. Oxford University Press.

Duvick, D. N. (1992) Genetic contributions to advances in yield of United States maize. Maydica 37:69-79.

Giroux, M. J., Shaw, J., Barry, G., Cobb, B. G., Greene, T., Okita, T., and Hannah, L. C. (1996) A single gene mutation that increases seed weight. Proc. Natl. Acad. Sci. USA 93:5824-5829.

Hannah, L. C., Giroux, M., and Boyer, C. D. (1993) Biotechnological modification of carbohydrates for sweet corn and maize improvement. Biotechnol. Hortic. Sci. Hortic. 55:177-197.

King, R. W., Wardlaw, I. F., and Evans, L. T. (1967) Effect of assimilate utilization on photosynthetic rate in wheat. Planta 77:261-276.

Lanning, S. P. Talbert, L. E. McNeal, F. H. Alexander, W. L. McGuire, C. F. Bowman, H. Carlson, G. Jackson, G. Eckhoff, J. Kushnak, G. (1992) Registration of ‘Hi-Line’ wheat. Crop Science 32:283-284.

Martinez-Carrasco, R., and Thorne, G. N. (1979) Effects of crop thinning and reduced grain numbers per ear on grain size in two winter wheat varieties given different amounts of nitrogen. Ann. Appl. Biol. 92:383-393.

Sakulsingharoj, C., Choi, S-B., and Okita, T. W. (1999) Increasing rice productivity by molecular genetic manipulation of ADP-glucosepyrophosphorylase in starch biosynthesis. General Meeting of the International Program on Rice Biotechnology.

Stark, D. M., Timmerman, K, Barry, G. F., Preiss, J., and Kishore, G. M. (1992) Regulation of the amount of starch in plant tissues by ADPglucose pyrophosphorylase. Science 258:287-292.

Sun, J., Okita, T. W., and Edwards, G. E. (1999) Modification of carbon partitioning, photosynthetic capacity, and 02 sensitivity in Arabidopsis plants with low ADP-glucose pyrophosphorylase activity. Plant Physiology 119:267-276.

Zamski, E., and Grunberger, Y. (1995) Short- and long-eared high-yielding hexaploid wheat cultivars: which has unexpressed potential for higher yield? Annals of Botany 75:501-506.

Anderson, J. M. et al. (1989) The Encoded Primary Sequence of a Rice Seed ADP-glucose Pyrophosphorylase Subunit and its Homology to the Bacterial Enzyme. The Journal of Biology Chemistry 264(21):12238-12242.

Anderson, J. M. et al. (1991) Molecular characterization of the gene encoding a rice endosperm-specific ADPglucose pyrophosphorylase subunit and its developmental pattern of transcription. Gene 97:199-205.

Dickinson, D. B., and Preiss J. (1969) Presence of ADP-glucose Pyrophosphorylase in Shrunken-2 and Brittle-2 Mutants of Maize Endosperm. Plant Physiol. 44:1058-1062.

Muller-Rober, B. T. et al. (1990) One of two different ADP-glucose pyrophosphorylase genes from potato responds strongly to elevated levels of sucrose. Mol. Gen. Genet. 224:136-146.

Morell, M., M. Bloom, J. Preiss (1988) Affinity Labeling of the Allosteric Activator Site(s) of Spinach Leaf ADP-glucose Pyrophosphorylase. The Journal of Biological Chemistry 263(2):633-637.

Nakata, Paul A. et al. (1991) Comparison of the primary sequences of two potato tuber ADP-glucose pyrophosphorylase subunits. Plant Molecular Biology 17:1089-1093.

Tsai, Chia-Yin, Oliver E. Nelson (1966) Starch-Deficient Maize Mutant Lacking Adenosine Diphosphate Glucose Pyrophosphorylases Activity. Science 151:341-343.

Greene, T. W. and Hannah, L. C. (1998) Enhanced Stability of Maize Endosperm ADP-Glucose Pyrophosphorylase is Gained through Mutants that Alter Subunit Interactions. Proc. Natl. Acad. Sci. USA 95:13342-13347.

Abbate, P. E., Andrade, F. H., Lazaro, L., Bariffi, J. F., Berardocco, H. G., Inza, V. H., and Marturano, F. (1998) Grain Yield Increase in Recent Argentine Wheat Cultivars. Crop Science 38:1203-1209.

Bindraban, P. S., Sayre, K. D., and Solis-Moya, E. (1998) Identifying Factors that Determine Kernel Number in Wheat. Field Crops Research 58:223-234.

Evans, L. T., Wardlaw, I. F., and Fisher, R. A. (1975) Crop Physiology: Some Case Histories. Evans L. T., Edited. Cambridge, Cambridge University Press, 101-149.

Rogers S. G., Klee H. J., Horsch R. B., Farley R. T. 1987. Improved vectors for plant transformation: Expression cassette vectors and new selectable markers. Methods in Enzymol. 153:253-277.

Sivamani, E., Shen, P., Opalka, N., Beachy, R. N., and Fauquet, C. M. 1996. Selection of large quantities of embryogenic calli from indica rice seeds for production of fertile transgenic plants using the biolistic method. Plant Cell Reports 15:322-327.

Spiertz, J. H. J., van Keulen, H. (1980) Effects of Nitrogen and Water Supply on Growth and Grain Yield of Wheat. Proceedings of the Third International Wheat Conference. May 22-June 3, Madrid, Spain.

Thorne, G. N., and Wood, D. W. (1987) Effects of Radiation and Temperature on Tiller Survival, Grain Number and Grain Yield in Winter Wheat. Annals of Botany 59:413-426.

Vasil, V., Srivastava, V., Castillo, A. M., Fromm, M. E., and Vasil, I. K. 1993. Rapid production of transgenic wheat plants by direct bombardment of cultured immature embryos. Bio/Technology 11:1553-1558.

Weeks, J. T., Anderson, O. D., and Blechl, A. E. 1993. Rapid production of multiple independent lines of fertile transgenic wheat (Triticum aestivum). Plant Physiol. 102:1077-1084.

Whingwiri, E. E., Kuo, J., and Stern, W. R. (1981) The Vascular System in the Rachis of a Wheat Ear. Annals of Botany 48:189-201.

Johnson, C. W., Carnahan, H. L., Tseng, S. T., Oster, J. J., and Hill, J. E. 1986. Registration of ‘M-202’ rice. Crop Science 26:198.

Fromm, M. E., Morrish, F. Armstrong, C. Williams, R. Thomas, J., and Klein, T. M. 1990. Inheritance and expression of chimeric genes in the progeny of transgenic maize plants. Bio/Technology 8:833-839.

I claim:

1. A method of increasing the number of seeds produced by a plant, wherein the method comprises:
a. introducing into a plant a nucleic acid, wherein the nucleic acid is selected from the group consisting of:
a nucleic acid comprising SEQ ID NO: 1, or a fragment of SEQ ID NO: 1 encoding a polypeptide that retains biological activity substantially the same as SH2−REV6;
a nucleic acid which hybridizes with SEQ ID NO: 1 under high stringency conditions and encodes a polypeptide that retains biological activity substantially the same as SH2−REV6;
a nucleic acid encoding a polypeptide comprising SEQ ID NO: 2, an allelic variant of SEQ ID NO: 2, a sequence which has at least 65% sequence identity to the relevant portion of SEQ ID NO: 2, or a fragment of said polypeptide that retains biological activity substantially the same as SH2−REV6;
a nucleic acid comprising SEQ ID NO: 3, or a fragment of SEQ ID NO: 3 encoding a polypeptide that retains biological activity substantially the same as SH2−REV6-HS;
a nucleic acid which hybridizes with SEQ ID NO: 3 under high stringency conditions and encodes a polypeptide that retains biological activity substantially the same as SH2−REV6-HS; and, a nucleic acid encoding a polypeptide comprising SEQ ID NO: 4, an allelic variant of SEQ ID NO: 4, a sequence which has at least 65% sequence identity to the relevant portion of SEQ ID NO: 4, or a fragment of said polypeptide that retains biological activity substantially the same as SH2−REV6-HS; and
b. growing the plant produced in step a, wherein said plant produces an increased number of seeds relative to a plant of the same genetic background in which said nucleic acid has not been introduced.

2. The method according to claim 1, wherein the plant is a monocotyledonous plant.

3. The method according to claim 2, wherein the plant is a plant selected from the group consisting of rice, wheat, barley, maize, and lilies.

4. The method according to claim 3, wherein the plant is wheat.

5. The method according to claim 3, wherein the plant is maize.

6. The method according to claim 3, wherein the plant is rice.

7. The method according to claim 1, wherein the plant is a dicotyledonous plant.

8. The method according to claim 7, wherein the plant is a plant selected from the group consisting of tobacco, soybeans, potato, sweet potato, radish, cabbage, rape, pea, and apple trees.

9. The method according to claim 8, wherein the plant is a pea plant.

10. The method according to claim 1, further comprising crossing the plant obtained in step b with a second plant and harvesting and growing the seed which is produced as a result of making the cross.

11. The method according to claim 1, further comprising harvesting seed which is produced by selfing the plant obtained in step b and growing the harvested seed.

12. The method according to claim 1, wherein said nucleic acid is operably linked to a promoter.

13. A method of increasing the biomass produced by a plant, wherein the method comprises:
a. introducing into a plant a nucleic acid, wherein the nucleic acid is selected from the group consisting of:
a nucleic acid comprising SEQ ID NO: 1, or a fragment of SEQ ID NO: 1 encoding a polypeptide that retains biological activity substantially the same as SH2−REV6;
a nucleic acid which hybridizes with SEQ ID NO: 1 under high stringency conditions and encodes a polypeptide that retains biological activity substantially the same as SH2−REV6;
a nucleic acid encoding a polypeptide comprising SEQ ID NO: 2, an allelic variant of SEQ ID NO: 2, a sequence which has at least 65% sequence identity to the relevant portion of SEQ ID NO: 2, or a fragment of said polypeptide that retains biological activity substantially the same as SH2−REV6;
a nucleic acid comprising SEQ ID NO: 3, or a fragment of SEQ ID NO: 3 encoding a polypeptide that retains biological activity substantially the same as SH2−REV6-HS;
a nucleic acid which hybridizes with SEQ ID NO: 3 under high stringency conditions and encodes a polypeptide that retains biological activity substantially the same as SH2−REV6-HS; and,
a nucleic acid encoding a polypeptide comprising SEQ ID NO: 4, an allelic variant of SEQ ID NO: 4, a sequence which has at least 65% sequence identity to the relevant portion of SEQ ID NO: 4, or a fragment of said polypeptide that retains biological activity substantially the same as SH2−REV6-HS; and
b. growing the plant produced in step a, wherein said plant produces increased biomass relative to a plant of the same genetic background in which said nucleic acid has not been introduced.

14. The method according to claim 13, wherein the plant is a monocotyledonous plant.

15. The method according to claim 14, wherein the plant is a plant selected from the group consisting of rice, wheat, barley, maize, and lilies.

16. The method according to claim 15, wherein the plant is wheat.

17. The method according to claim 15, wherein the plant is maize.

18. The method according to claim 15, wherein the plant is rice.

19. The method according to claim 13, wherein the plant is a dicotyledonous plant.

20. The method according to claim 19, wherein the plant is a plant selected from the group consisting of tobacco, soybeans, potato, sweet potato, radish, cabbage, rape, pea, and apple trees.

21. The method according to claim 20, wherein the plant is a pea plant.

22. The method according to claim 13, further comprising crossing the plant obtained in step b with a second plant and harvesting and growing the seed which is produced as a result of making the cross.

23. The method according to claim 13, further comprising harvesting seed which is produced by selfing the plant obtained in step b and growing the harvested seed.

24. The method according to claim 13, wherein said nucleic acid is operably linked to a promoter.

25. A method of increasing the Harvest Index of a plant, wherein the method comprises:
a. introducing into a plant a nucleic acid, wherein the nucleic acid is selected from the group consisting of:
a nucleic acid comprising SEQ ID NO: 1, or a fragment of SEQ ID NO: 1 encoding a polypeptide that retains biological activity substantially the same as SH2−REV6;
a nucleic acid which hybridizes with SEQ ID NO: 1 under high stringency conditions and encodes a polypeptide that retains biological activity substantially the same as SH2−REV6;
a nucleic acid encoding a polypeptide comprising SEQ ID NO: 2, an allelic variant of SEQ ID NO: 2, a sequence which has at least 65% sequence identity to the relevant portion of SEQ ID NO: 2, or a fragment of said polypeptide that retains biological activity substantially the same as SH2−REV6;
a nucleic acid comprising SEQ ID NO: 3, or a fragment of SEQ ID NO: 3 encoding a polypeptide that retains biological activity substantially the same as SH2−REV6-HS;
a nucleic acid which hybridizes with SEQ ID NO: 3 under high stringency conditions and encodes a polypeptide that retains biological activity substantially the same as SH2−REV6-HS; and,
a nucleic acid encoding a polypeptide comprising SEQ ID NO: 4, an allelic variant of SEQ ID NO: 4, a sequence which has at least 65% sequence identity to the relevant portion of SEQ ID NO: 4, or a fragment of said polypeptide that retains biological activity substantially the same as SH2−REV6-HS; and
b. growing the plant produced in step a, wherein said plant exhibits an increased Harvest Index relative to a plant of the same genetic background in which said nucleic acid has not been introduced.

26. The method according to claim 25, wherein the plant is a monocotyledonous plant.

27. The method according to claim 26, wherein the plant is a plant selected from the group consisting of rice, wheat, barley, maize, and lilies.

28. The method according to claim 27, wherein the plant is wheat.

29. The method according to claim 27, wherein the plant is maize.

30. The method according to claim 27, wherein the plant is rice.

31. The method according to claim 25, wherein the plant is a dicotyledonous plant.

32. The method according to claim 31, wherein the plant is a plant selected from the group consisting of tobacco, soybeans, potato, sweet potato, radish, cabbage, rape, pea, and apple trees.

33. The method according to claim 32, wherein the plant is a pea plant.

34. The method according to claim 25, further comprising crossing the plant obtained in step b with a second plant and harvesting and growing the seed which is produced as a result of making the cross.

35. The method according to claim 25, further comprising harvesting seed which is produced by selfing the plant obtained in step b and growing the harvested seed.

36. The method according to claim 25, wherein said nucleic acid is operably linked to a promoter.

37. A method of increasing the flag leaf weight of a monocotyledonous plant, wherein the method comprises:
a. introducing into a plant a nucleic acid, wherein the nucleic acid is selected from the group consisting of:
a nucleic acid comprising SEQ ID NO: 1, or a fragment of SEQ ID NO: 1 encoding a polypeptide that retains biological activity substantially the same as SH2−REV6;
a nucleic acid which hybridizes with SEQ ID NO: 1 under high stringency conditions and encodes a polypeptide that retains biological activity substantially the same as SH2−REV6;
a nucleic acid encoding a polypeptide comprising SEQ ID NO: 2, an allelic variant of SEQ ID NO: 2, a sequence which has at least 65% sequence identity to the relevant portion of SEQ ID NO: 2, or a fragment of said polypeptide that retains biological activity substantially the same as SH2−REV6;
a nucleic acid comprising SEQ ID NO: 3, or a fragment of SEQ ID NO: 3 encoding a polypeptide that retains biological activity substantially the same as SH2−REV6-HS;
a nucleic acid which hybridizes with SEQ ID NO: 3 under high stringency conditions and encodes a polypeptide that retains biological activity substantially the same as SH2−REV6-HS; and,
a nucleic acid encoding a polypeptide comprising SEQ ID NO: 4, an allelic variant of SEQ ID NO: 4, a sequence which has at least 65% sequence identity to the relevant portion of SEQ ID NO: 4, or a fragment of said polypeptide that retains biological activity substantially the same as SH2−REV6-HS; and
b. growing the plant produced in step a, wherein said plant exhibits increased flag leaf weight relative to a plant of the same genetic background in which said nucleic acid has not been introduced.

38. The method according to claim 37, further comprising crossing the plant obtained in step b with a second plant and harvesting and growing the seed which is produced as a result of making the cross.

39. The method according to claim 37, further comprising harvesting seed which is produced by selfing the plant obtained in step b and growing the harvested seed.

40. The method according to claim 37, wherein said nucleic acid is operably linked to a promoter.

41. A method of increasing the number of seed heads produced by a monocotyledonous plant, wherein the method comprises:
a. introducing into a plant a nucleic acid, wherein the nucleic acid is selected from the group consisting of:
a nucleic acid comprising SEQ ID NO: 1, or a fragment of SEQ ID NO: 1 encoding a polypeptide that retains biological activity substantially the same as SH2−REV6;
a nucleic acid which hybridizes with SEQ ID NO: 1 under high stringency conditions and encodes a polypeptide that retains biological activity substantially the same as SH2−REV6;
a nucleic acid encoding a polypeptide comprising SEQ ID NO: 2, an allelic variant of SEQ ID NO: 2, a sequence which has at least 65% sequence identity to the relevant portion of SEQ ID NO: 2, or a fragment of said polypeptide that retains biological activity substantially the same as SH2−REV6;
a nucleic acid comprising SEQ ID NO: 3, or a fragment of SEQ ID NO: 3 encoding a polypeptide that retains biological activity substantially the same as SH2−REV6-HS;
a nucleic acid which hybridizes with SEQ ID NO: 3 under high stringency conditions and encodes a polypeptide that retains biological activity substantially the same as SH2−REV6-HS; and, a nucleic acid encoding a polypeptide comprising SEQ ID NO: 4, an allelic variant of SEQ ID NO: 4, a sequence which has at least 65% sequence identity to the relevant portion of SEQ ID NO: 4, or a fragment of said polypeptide that retains biological activity substantially the same as SH2−REV6-HS; and
b. growing the plant produced in step a, wherein said plant produces an increased number of seed heads relative to a plant of the same genetic background in which said nucleic acid has not been introduced.

42. The method according to claim 41, further comprising crossing the plant obtained in step b with a second plant and harvesting and growing the seed which is produced as a result of making the cross.

43. The method according to claim 41, further comprising harvesting seed which is produced by selfing the plant obtained in step b and growing the harvested seed.

44. The method according to claim 41, wherein said nucleic acid is operably linked to a promoter.

45. A method of increasing two or more traits of a dicotyledonous plant, wherein the traits are selected from the group consisting of the number of seeds, average seed weight, total seed weight, number of seed heads, Harvest Index and total plant weight, wherein the method comprises:
a. introducing into a plant a nucleic acid, wherein the nucleic acid is selected from the group consisting of:
a nucleic acid comprising SEQ ID NO: 1, or a fragment of SEQ ID NO: 1 encoding a polypeptide that retains biological activity substantially the same as SH2−REV6;
a nucleic acid which hybridizes with SEQ ID NO: 1 under high stringency conditions and encodes a polypeptide that retains biological activity substantially the same as SH2−REV6;
a nucleic acid encoding a polypeptide comprising SEQ ID NO: 2, an allelic variant of SEQ ID NO: 2, a sequence which has at least 65% sequence identity to the relevant portion of SEQ ID NO: 2, or a fragment of said polypeptide that retains biological activity substantially the same as SH2−REV6;
a nucleic acid comprising SEQ ID NO: 3, or a fragment of SEQ ID NO: 3 encoding a polypeptide that retains biological activity substantially the same as SH2−REV6-HS;
a nucleic acid which hybridizes with SEQ ID NO: 3 under high stringency conditions and encodes a polypeptide that retains biological activity substantially the same as SH2−REV6-HS; and,
a nucleic acid encoding a polypeptide comprising SEQ ID NO: 4, an allelic variant of SEQ ID NO: 4, a sequence which has at least 65% sequence identity to the relevant portion of SEQ ID NO: 4, or a fragment of said polypeptide that retains biological activity substantially the same as SH2−REV6-HS; and
b. growing the plant produced in step a, wherein said plant exhibits an increase in said two or more traits relative to a plant of the same genetic background in which said nucleic acid has not been introduced.

46. The method according to claim 45, further comprising crossing the plant obtained in step b with a second plant and harvesting and growing the seed which is produced as a result of making the cross.

47. The method according to claim 45, further comprising harvesting seed which is produced by selfing the plant obtained in step b and growing the harvested seed.

48. The method according to claim 45, wherein said nucleic acid is operably linked to a promoter.

46. A method of increasing the yield of two or more traits of a monocotyledonous plant, wherein the traits are selected from the group consisting of the number of seeds, average seed weight, total seed weight, number of seed heads, flag leaf weight, Harvest Index and total plant weight, wherein the method comprises:
a. introducing into a plant a nucleic acid, wherein the nucleic acid is selected from the group consisting of:
a nucleic acid comprising SEQ ID NO: 1, or a fragment of SEQ ID NO: 1 encoding a polypeptide that retains biological activity substantially the same as SH2−REV6;
a nucleic acid which hybridizes with SEQ ID NO: 1 under high stringency conditions and encodes a polypeptide that retains biological activity substantially the same as SH2−REV6;
a nucleic acid encoding a polypeptide comprising SEQ ID NO: 2, an allelic variant of SEQ ID NO: 2, a sequence which has at least 65% sequence identity to the relevant portion of SEQ ID NO: 2, or a fragment of said polypeptide that retains biological activity substantially the same as SH2−REV6;
a nucleic acid comprising SEQ ID NO: 3, or a fragment of SEQ ID NO: 3 encoding a polypeptide that retains biological activity substantially the same as SH2−REV6-HS;
a nucleic acid which hybridizes with SEQ ID NO: 3 under high stringency conditions and encodes a polypeptide that retains biological activity substantially the same as SH2−REV6-HS; and,
a nucleic acid encoding a polypeptide comprising SEQ ID NO: 4, an allelic variant of SEQ ID NO: 4, a sequence which has at least 65% sequence identity to the relevant portion of SEQ ID NO: 4, or a fragment of said polypeptide that retains biological activity substantially the same as SH2−REV6-HS; and
b. growing the plant produced in step a, wherein said plant exhibits an increase in said two or more traits relative to a plant of the same genetic background in which said nucleic acid has not been introduced.

47. The method according to claim 46, further comprising crossing the plant obtained in step b with a second plant and harvesting and growing the seed which is produced as a result of making the cross.

48. The method according to claim 46, comprising harvesting seed which is produced by selfing the plant obtained in step b and growing the harvested seed.

49. The method according to claim 46, wherein said nucleic acid is operably linked to a promoter.


SEQUENCE LISTING (6 sequences)

SEQ ID NO: 1 - DNA, 1582 bp, Zea mays
  CDS (10)..(1563): Shrunken-2 gene revertant form
ggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgckatcattttgggcggaggcactggatctcagctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatactcaattacatgactttggatctgaaatcctcccaagagctgtactagatcatagtgtgcaggcatgcatttttacgggctattgggaggatgttggaacaatcaaatcattctttgatgcaaacttggccctcactgagcagccttccaagtttgatttttacgatccaaaaacacctttcttcactgcaccccgatgcttgcctccgacgcaattggacaagtgcaagatgaaatatgcatttatctcagatggttgcttactgagagaatgcaacatcgagcattctgtgattggagtctgctcacgtgtcagctctggatgtgaactcaaggactccgtgatgatgggagcggacatctatgaaactgaagaagaagcttcaaagctactgttagctgggaaggtcccrgttggaataggaaggaacacaaagataaggaactgtatcattgacatgaatgctaggattgggaagaacgtggtgatcacaaacagtaagggcatccaagaggctgatcacccggaagaagggtactcgtactacataaggtctggaatcgtggtgatcctgaagaatgcaaccatcaacgatgggtctgtcatatagatcggctgcgtktgcg

SEQ ID NO: 2 - protein, 518 aa, Zea mays
MQFALALDTNSGPHQIRSCEGDGIDRLEKLSIGGRKQEKALRNRCFGGRVAATTQCILTSDACPETLHSQTQSSRKNYADANRVSAIILGGGTGSQLFPLTSTRATPAVPVGGCYRLIDIPMSNCFNSGINKIFVMSQFNSTSLNRHIHRTYLEGGINFADGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYTQLHDFGSEILPRAVLDHSVQACIFTGYWEDVGTIKSFFDANLALTEQPSKFDFYDPKTPFFTAPRCLPPTQLDKCKMKYAFISDGCLLRECNIEHSVIGVCSRVSSGCELKDSVMMGADIYETEEEASKLLLAGKVPVGIGRNTKIRNCIIDMNARIGKNVVITNSKGIQEADHPEEGYSYYIRSGIVVILKNATINDGSVI

SEQ ID NO: 3 - DNA, 1582 bp, Zea mays
  CDS (10)..(1563): Shrunken-2 gene revertant form, modified to be heat stable
ggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgckatcattttgggcggaggcactggatctcagctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatactcaattacatgactttggatctgaaatcctcccaagagctgtactagattayagtgtgcaggcatgcatttttacgggctattgggaggatgttggaacaatcaaatcattctttgatgcaaacttggccctcactgagcagccttccaagtttgatttttacgatccaaaaacacctttcttcactgcaccccgatgcttgcctccgacgcaattggacaagtgcaagatgaaatatgcatttatctcagatggttgcttactgagagaatgcaacatcgagcattctgtgattggagtctgctcacgtgtcagctctggatgtgaactcaaggactccgtgatgatgggagcggacatctatgaaactgaagaagaagcttcaaagctactgttagctgggaaggtcccrgttggaataggaaggaacacaaagataaggaactgtatcattgacatgaatgctaggattgggaagaacgtggtgatcacaaacagtaagggcatccaagaggctgatcacccggaagaagggtactcgtactacataaggtctggaatcgtggtgatcctgaagaatgcaaccatcaacgatgggtctgtcatatagatcggctgcgtktgcg

SEQ ID NO: 4 - protein, 518 aa, Zea mays
MQFALALDTNSGPHQIRSCEGDGIDRLEKLSIGGRKQEKALRNRCFGGRVAATTQCILTSDACPETLHSQTQSSRKNYADANRVSAIILGGGTGSQLFPLTSTRATPAVPVGGCYRLIDIPMSNCFNSGINKIFVMSQFNSTSLNRHIHRTYLEGGINFADGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYTQLHDFGSEILPRAVLDYSVQACIFTGYWEDVGTIKSFFDANLALTEQPSKFDFYDPKTPFFTAPRCLPPTQLDKCKMKYAFISDGCLLRECNIEHSVIGVCSRVSSGCELKDSVMMGADIYETEEEASKLLLAGKVPVGIGRNTKIRNCIIDMNARIGKNVVITNSKGIQEADHPEEGYSYYIRSGIVVILKNATINDGSVI

SEQ ID NO: 5 - DNA, 26 bp, Artificial Sequence
  Description of Artificial Sequence  PCR primer
ctggatgtgaactcaaggactccgtg

SEQ ID NO: 6 - DNA, 24 bp, Artificial Sequence
  Description of Artificial Sequence  PCR primer
ggcttaactatgcggcatcagagc